(19) United States
(12) Patent Application Publication
(10) Pub. No.: US 2007/0155682 A1
(43) Pub. Date: Jul. 5, 2007

(54) Translational control by small, non-translatable rnas

(76) Inventor: Henri Tiedge, New York, NY (US)

Correspondence Address: DILWORTH & BARRESE, LLP, 333 EARLE OVINGTON BLVD., SUITE 702, UNIONDALE, NY, 11553, US

(21) Appl. No.: 10/534,789

(22) PCT Filed: Nov. 12, 2003
(86) PCT No.: PCT/US03/35897

§ 371 Date: Oct. 11, 2005

Related U.S. Application Data
(60) Provisional application No. 60/425,475, filed on Nov. 12, 2002.

(51) Int. Cl. A61K 48/00; C07H 21/02

(52) U.S. Cl. 514/44; 536/23.1

(57) ABSTRACT

The present invention provides isolated antisense molecules targeted to BC200 RNA. The subject antisense molecules are useful in treating various neurological disorders and carcinomas in a subject Also provided are methods of treating a neurological disorder or cancer in a subject by administering a therapeutically effective amount of a subject antisense molecule. A method for treating epilepsy in a patient by administering an effective amount of BC200 RNA is also provided. Further, kits comprising a subject antisense molecule and a pharmaceutically acceptable carrier are provided by the present invention.

### CROSS REFERENCE TO RELATED APPLICATIONS

This application claims benefit of U.S. Provisional Application Ser. No. 60/425,475, filed Nov. 12, 2002, and incorporated by reference herein.

### GOVERNMENT RIGHTS

This invention was made with Government support under National Institutes of Health Grant NS 13458. The Government has certain rights in the invention

### BACKGROUND OF THE INVETION

In neurons, local protein synthesis in synaptodendritic microdomains has been implicated in the growth and plasticity of synapses. Prerequisites for local translation are the targeted transport of RNAs to distal sites of synthesis in dendrites, and translational control mechanisms to limit synthesis to times of demand. Translational control in neurons is also implicated in the development of certain neurological disorders.

Diverse types of neuronal mRNAs are transported to distal target sites such as postsynaptic dendritic microdomains where they are presumed to be translated into cognate proteins on-site (for reviews, see Kindler et al., 1997; Tiedge et al., 1999; Kiebler and DesGroseillers, 2000; Wells et al., 2000; Greenough et al., 2001; Job and Eberwine, 2001b; Richter, 2001; Steward and Schuman, 2001). Characterized by highly elongated dendritic and axonal processes that form large numbers of synaptic connections, nerve cells have been suggested to rely on local protein synthesis for an effective management of their mosaic postsynaptic protein repertoires in dendrites. Experience-dependent, site-specific modulations of synaptic protein complements through local synthesis are thus thought to provide a BASIS for long-lasting plastic changes of synaptic form and function (Tiedge et al., 1999; Job and Eberwine, 2001b).

The notion of postsynaptic translation has in recent years been strengthened by the discovery of various neuronal RNAs that are selectively localized to dendrites. Dendritic mRNAs encode proteins that belong to different classes, including cytosolic proteins, cytoskeletal components, as well as membrane-associated and membrane-integrated proteins (for reviews, see Kiebler and DesGroseillers, 2000; Job and Eberwine, 2001b; Richter, 2001). According to a recent estimate (Eberwine et al., 2001), the family of dendritic mRNAs is comprised of several hundred members.

Components of the translational machinery have been identified in dendritic domains (Tiedge and Brosius, 1996; Torre and Steward, 1996; Gardiol et al., 1999). Dendritic translation has been documented in physically isolated dendrites (Torre and Steward, 1992) and in cultured neurons (Crino and Eberwine, 1996). Local translation has also been shown to be a requirement for synapse formation (Schacher and Wu, 2002). Recent data further suggest that protein synthesis in dendrites can be subject to modulation by neuronal activity, receptor activation, and neurotrophic action (Steward and Halpain, 1999; Kacharmina et al., 2000; Scheetz et al., 2000; Aakalu et al., 2001; Greenough et al., 2001; Job and Eberwine, 2001a). The available evidence, in summary, is in support of a model in which a select group of mRNAs is transported to dendrites, subsequent to which they can be translated, upon demand, in specific postsynaptic microdomains where the cognate proteins are required Tiedge et al., 1999; Job and Eberwine, 2001b).

This model, while attractive, relies on a number of premises that have not been addressed. Paramount among them is the issue of translational control. To prevent inappropriate protein synthesis at the wrong place or at the wrong time, the translational activity of any dendritic mRNA will have to be tightly controlled during the sequential steps of targeted transport, postsynaptic localization, and regulated local translation (Job and Eberwine, 2001b). A key question in this regard is raised by the assumption that many dendritic mRNAs may remain translationally silent after they have reached their postsynaptic target sites, until such time that an appropriate signal is received.

BC200 RNA is a 200-nucleotide long, non-translatable RNA that is prevalently expressed in the nervous system of primates, including man. A partial nucleotide sequence of BC200 RNA from monkey brains was reported by Watson and Sutcliffe, Molecular & Cellular Biology 7, 3324-3327 (1987). This 138 nucleotide sequence showed substantial homology to the Alu left monomer, a sequence that is repeated many times throughout the human and other primate genomes. BC200 RNA does not normally occur in detectable amounts in normal non-neuronal tissue other than germ cells, but does occur in high amounts in a variety of non-neuronal human tumor tissues.

The primary sequence of BC200 RNA can be subdivided into three structural domains. Domain I is nucleotides 1-122 and is substantially homologous to Alu repetitive elements which are found in high copy numbers in primate genomes. However, this region includes two bases not found in Alu or SRP-RNA, i.e., nucleotides at positions 48 and 49, which can be used to develop amplification primers specific to BC200 sequences. Domain II is an A-rich region consisting of nucleotides 123-158. Domain III, consisting of nucleotides 159-200, contains a unique sequence with no homology to other known human sequences which can be used to identify BC200 RNA in tissues.

U.S. Pat. No. 5,670,318, the contents of which are incorporated herein by reference as if fully set forth, discloses the complete sequence of human BC200 RNA and the use of polynucleotide probes which can be used to specifically detect the presence of human BC200 RNA in human breast tissue as an indicator of breast adenocarcinoma. U.S. Pat. No. 5,736,329, the contents of which are incorporated herein by reference as if fully set forth, discloses the use of polynucleotide probes which can be used to specifically detect the presence of human BC200 RNA in human brain tissue as an indicator of Alzheimer's Disease.

In accordance with the present invention, it has now been discovered that BC200 RNA and BC1 RNA (the rodent counterpart to BC200 RNA) are specific repressors of translation initiation in both cap-dependent and internal entry modes. Therefore, nontranslatable BC1 and BC200 RNA play a functional role in translational control of gene expression in neurons. It has also been discovered in accordance with the present invention, that BC1 RNA levels are down-regulated in response to the induction of epileptiform activity. Thus, the present invention provides oligonucleotides which may be used as antisense molecules to reduce BC200 RNA levels in various carcinomas and neuronal disorders. In addition, the present invention provides methods of treating patients suffering from various carcinomas and neuronal disorders by down-regulating levels of BC200 RNA transcripts in such patients. The present invention also provides methods for treating patients suffering from epilepsy by up-regulating levels of BC200 RNA transcripts.

### SUMMARY OF THE INVENTION

The present invention provides isolated antisense molecules targeted to BC200 RNA. In particular, there are provided isolated antisense molecules comprising a nucleotide sequence targeted to the sequence set forth in SEQ ID NO:1 and/or SEQ ID NO:2.

Specific antisense molecules provided by the present invention comprise the nucleotide sequences set forth in SEQ ID NO:3, SEQ ID NO:4, SEQ ID NO:5, or SEQ ID NO:6. Pharmaceutical compositions comprising at least one subject antisense molecule or BC200 RNA transcript admixed with a pharmaceutical acceptable carrier are also provided.

The present invention further provides a method for treating a neurological disorder or cancer in a subject. The method comprises down-regulating BC200 RNA in the subject. The down-regulating of BC200 RNA in a subject may comprise administering a therapeutically effective amount of a dominant negative mutant of BC200 RNA or a small interfering RNA. In addition, the down-regulating of BC200 RNA may comprise administering a therapeutically effective amount of an antisense molecule targeted to the nucleotide sequence set forth in SEQ ID NO:1 or SEQ ID NO:2. In another embodiment of the invention, the down-regulating of BC200 comprises administering a therapeutically effective amount of at least one of SEQ ID NO:3, SEQ ID NO:4, SEQ ID NO:5, or SEQ ID NO:6. Examples of neurological disorders which may be treated by the methods of the invention include, but are not limited to Alzheimer's disease, Fragile X mental retardation syndrome, Down's syndrome and Parkinson's disease.

Examples of cancer which may be treated by the present invention include but are not limited to squamous cell carcinoma of the tongue and lung, epithelial carcinoma of the esophagus, tubular adenocarcinoma of the stomach, breast adenocarcinoma, adenocarcinoma of the lung, mucoepidermoid of the partoid gland, melanoma of the skin, papillary carcinoma of the ovaries, or endothelial adenocarcinoma of the cervix.

In still another aspect of the invention, there is provided a method for treating epilepsy in a subject The method comprises up-regulating BC200 RNA in a patient Examples of up-regulating in this context comprises administering to the patient a therapeutically effective amount of BC200 RNA or a gene therapy construct having a DNA or RNA corresponding to BC200 operably linked to a promoter which functions in the cells of the subject.

The present invention also provides kits which comprise at least one subject antisense molecule and a pharmaceutically acceptable carrier.

### BRIEF DESCRIPTION OF THE FIGURES

FIG. 1A, 1B, 1C and 1D illustrate the effects of BC1 RNA as a repressor of translation in the sub-micromolar concentration range through the use of phosphorimaging. Protein products were labeled by ³⁵S-methionine incorporation, using the RRL system, and were visualized by SDS PAGE and autoradiography. As seen in FIG. 1A, translation of endogenous RRL mRNAs was inhibited by increasing concentrations of BC1 RNA. Relative signal intensities of the major band were quantified by phosphorimaging and are listed for each lane. The signal intensity generated in the absence of BC1 RNA was assigned a relative value of 1. FIG. 1B presents the results from 3 experiments, quantified by phosphorimaging, showing that the signal of the major protein band was reduced by 72% at 320 nM BC1 RNA (one-way ANOVA, P<0.001; Scheffe's multiple comparison post hoc analysis (comparison with 0 nM BC1 RNA control), P<0.01 (**) for 40 nM BC1 RNA, P<0.001 (***) for other groups). Signal intensities of other protein bands were similarly reduced by 70-80%. Note that the x-axis is exponential. As shown in FIG. 1C, no inhibition of translation was observed in the presence of control RNAs, including U4 and U6 snRNAs, and tRNAs. As set forth in FIG. 1D, when capped and polyadenylated a-tubulin MRNA was used as a programming mRNA, translation was similarly inhibited in the same BC1 concentration range. Each experiment shown in C and D was performed at least twice.

FIG. 2A is a schematic diagram summarizing the steps in translation initiation that lead to the successive formation of 48S and 80S complexes. Steps that are targeted by inhibitors GMP-PNP and cycloheximide are indicated by arrows. The heterotrimeric complex eIF4F consists of eIF4A, eIF4E, and eIF4G. The helicase activity of eIF4A is stimulated by eIF4B. In addition, eIF4A is also present in free, monomeric form. (For more detailed diagrams of the translation initiation pathway, see Gingras et al., 1999; Hershey and Merrick, 2000; Dever, 2002.)

FIGS. 2B, 2C, 2D and 2E are graphic depictions of the effects of cycloheximide and GMP-PNP on translation initiation and the fact that BC1 RNA inhibits 48S and 80S complex assembly in cap-dependent initiation. As set forth in FIG. 2B, ³²P-labeled capped and polyadenylated α-tubulin mRNA was used as a programming mRNA in the presence of cycloheximide to visualize 80S complexes. At 600 nM BC1 RNA, 80S complex formation was found to be reduced by 61%±5% (measured from the slope of the ribonucleoprotein complex peak; 3 experiments). As shown in FIG. 2C, analogously, assembly of 48S preinitiation complexes was visualized by using GMP-PNP. At 600 nM BC1 RNA, 48S complex formation was found to be reduced by 81%±5% (measured from the slope of the ribonucleoprotein complex peak; 3 experiments). FIG. 2D demonstrates that, in contrast to BC1 RNA, U4 RNA at the same concentration had no effect on 48S complex assembly. FIG. 2E establishes that formation of 48S complexes on non-adenylated α-tubulin programming niRNA was inhibited in the presence of BC1 RNA to an extent similar to polyadenylated α-tubulin mRNA (compare with FIG. 2C). Assembled complexes were resolved by sucrose density gradient centrifugation. Sedimentation was from right to left Fractions from upper parts of the gradient have been omitted for clarity. Tub(A) mRNA, polyadenylated (A₉₈) α-tubulin mRNA; Tub mRNA, non-adenylated α-tubulin mRNA.

FIGS. 3A, 3B, 3C and 3D illustrate phosphorimaging results demonstrating BC1 RNA inhibition of translation initiated by the EMCV IRES. FIGS. 3A, 3C, and 3D are original gels; FIG. 3B graphically depicts combined results from phosphorimaging of 6 gels, one of which is shown as a representative example in FIG. 3A. In FIG. 3A, the programming mRNA encoded GFP, contained an EMCV IRES in the 5′ untranslated region (UTR), was used uncapped. FIG. 3B presents the results from 6 experiments, quantified by phosphorimaging, showing that translation was repressed by 79% at 320 nM BC1 RNA (one-way ANOVA, P<0.001; Scheffe's multiple comparison post hoc analysis (comparison with 0 nM BC1 RNA control), P<0.001 (***) for all groups). As set forth in FIG. 3C, as a control, the same mRNA was translated in the presence of U4 RNA. FIG. 3D demonstrates that both cap-initiated and IRES-initiated translation from a dicistronic programming mRNA were repressed by BC1 RNA. The first, cap-dependent cistron encoded Blue Fluorescent Protein (BFP). An EMCV IRES preceded the second, GFP-encoding cistron.

FIGS. 4A and 4C are examples of gels obtained for phosphorimaging analysis, the results of which are graphically depicted in FIG. 4B. FIG. 4D is a graphical depiction showing that translation and 48S complex formation mediated by the CSFV IRES are refractory to repression by BC1 RNA. The uncapped but polyadenylated programming mRNA encoded a truncated version of the influenza virus non-structural protein (NS′). As can be seen in FIGS. 4A and 4B, translation efficiency was not significantly altered by increasing concentrations of BC1 RNA (one-way ANOVA, P=0.9694, n=5). FIG. 4C demonstrates that nuclear U4 RNA also failed to affect translation initiated from the CSFV IRES. As shown in FIG. 4D, assembly of 48S complexes mediated by the CSFV IRES was refractory to inhibition by BC1 RNA (3 experiments). 48S complexes were assembled in the presence of GMP-PNP and were resolved by sucrose density gradient centrifugation as described above (see FIG. 2).

FIGS. 5A, 5B, 5C and 5D illustrate the binding activity of BC1 RNA to translational factors eIF4A and PABP. Electrophoresis Mobility Shift Assay (EMSA) experiments were performed with ³²P-labeled BC1 RNA As set forth in FIG. 5A, when BC1 RNA was incubated with eIF4A in the absence or presence of unlabeled competitor RNAs, unlabeled BC1 RNA, but not unlabeled random sequence (RS) RNA or tRNAs, competed for binding to eIF4A and effectively abolished the mobility shift. FIG. 5B demonstrates that BC1 RNA produced a band shift with full-length PABP. Effective competition was seen with unlabeled BC1 RNA, but not with unlabeled U4 RNA or U6 RNA. As shown in FIG. 5C, simultaneous incubation of BC1 RNA with eIF4A and PABP (N-terminal segment) produced a more substantial mobility shift than incubation with either protein alone. FIG. 5D establishes that, in rat brain extracts, BC1 RNA was observed to be shifted to two bands of lower mobility (lane 1). An antibody specific for PABP (lane 2), but not a control antibody against GST (lane 3), produced a supershift with BC1 RNA. Conversely, the regular mobility shift of BC1 RNA was reduced in brain extracts that had been immunodepleted of PABP; note the reduction in intensity of the major BC1 RNA complex bands and the appearance of a band at higher mobility (lane 5). (BE, brain extract; ID BE, PABP-immunodepleted brain extract.)

FIGS. 6A, 6B, 6C and 6D are immunocytochemical results establishing that factors eIF4A, eIF4G, and PABP are enriched in synaptodendritic microdomains of hippocampal neurons in culture. Neurons were labeled (red fluorescence) for eIF4G FIG. 6A, for PABP FIG. 6B, or for eIF4A FIG. 6C. Cells were double-labeled with an antibody against synaptophysin (green fluorescence). Boxed dendritic segments are shown at 3-times higher magnification in insets. Note the clustered appearance of dendritic labeling signals for all three factors. Such clusters were often but not always observed in apposition to synaptophysin puncta. FIG. 6D presents the results of control experiments, which were performed in an identical manner except that incubation with primary antibodies was omitted. (Scale bar, 10 μm.)

FIG. 7 is a gel photograph demonstrating that human BC200 RNA inhibits translation in the same concentration range as its rodent counterpart BC1 RNA does. The programming mRNA used in these experiments was the EMCV-IRES/GFP mRNA that was also used in FIG. 3A/B. The results show that human BC200 RNA, like rodent BC1 RNA, is an effective repressor of translation if initiation is mediated by way of internal ribosome entry of the EMCV type.

FIG. 8 is a representative electrophysiological recording of an LTP experiment. Shown is the time course of LTP in the dentate gyrus. Animals were implanted unilaterally with stimulating and recording electrodes in the perforant path and dentate gyrus, respectively. The initial slope of the field EPSP was measured for each response. Averaged traces of pre- and posttetanic baselines are shown in insets. A 90 min stimulation was applied as indicated by diagonal lines. Posttetanic baseline was recorded for at least 30 min in each experiment, and maintenance of potentiation was verified immediately before fixation of brains.

FIGS. 9A-9D graphically depict expression of BC1 RNA after induction of LTP (as shown in FIG. 8). Numbers of animals used for each experiment are indicated (n). For each animal, 3-6 sections were examined, signal intensities were measured for selected areas, and means established for each area A-C, Diagrams present ratios of signal intensities of stimulated to unstimulated hippocampus in experimental groups (2 hrs LTP and 3 hrs LTP), or corresponding sides (left to right) in control groups (control). A, CA3 (stratum radiatum); B, CA1 (stratum radiatum and pyramidale); C, dentate gyrus (stratum moleculare). Ratios of signal intensities of CA3 stratum radiatum to CA3 stratum pyramidale in left (L) and right (R) hippocampi are shown in D (left side stimulated). Values are given as mean±sem. Analysis of variance (one-way ANOVA) revealed no significant differences between any of the compared areas (p>0.1 for A-D).

FIG. 10 is a representative electrophysiological recording of a kindling experiment. Shown is a hippocampal EEG that includes induction and development of an epileptic AD. Animals were implanted unilaterally with stimulating and recording electrodes in the stratum radiatum of CA3 and CA1, respectively. A 60 Hz train is followed by a 10 second AD. The lower panel shows the AD at a higher temporal resolution. The typical appearance of hippocampal epileptiform activity is evidenced by spikes displayed on depolarizing waves (spikes are clipped in this illustration).

FIGS. 11A-C are autoradiographs showing distribution of BC1 RNA and Arc mRNA after AD induction (as shown in FIG. 3). Labeling intensities are indicated by darkness of the autoradiographic signal. Brain areas shown include the mid-dorsal hippocampus. The right side was stimulated in all experiments. A, Expression of BC1 RNA after AD induction; B, expression of BC1 RNA in a control animal; C, expression of Arc mRNA after AD induction. Arc mRNA expression is strongly upregulated in the stimulated dentate gyrus (right hemisphere) but also shows some induction contralaterally (C). No significant expression of Arc mRNA was observed in unstimulated animals (not shown). In A, the puncture introduced by the stimulating electrode is indicated by an arrow. The line of reduced signal above the puncture in CA3 is produced by the physical insertion of the electrode through the neocortex. In the control animal (B), BC1 expression is higher in the right hemisphere than in the left hemisphere. After AD induction, BC1 expression levels in the stimulated (right side) hippocampus are similar to levels in the unstimulated (left) side (A). Scale bar, 800 μm

FIGS. 12A-D are histograms of BC1 expression and distribution in control and stimulated animals AD induction results in a significant reduction of somatodendritic BC1 levels in the CA3 region of the hippocampus. FIGS. 12A-12C show columns reflecting the signal ratios of stimulated to unstimulated hippocampus, or the corresponding sides (right to left) in control groups. Note that control animals express higher levels of BC1 RNA in the right hippocampus. While BC1 expression levels appear reduced ipsilaterally throughout the hippocampus in stimulated animals, such decrease was found statistically significant in stratum radiatum of CA3 (A). D shows signal ratios of CA3 stratum radiatum to CA3 stratum pyramidale for the unstimulated (left, L) and the stimulated (right, R) hippocampal side. Numbers of animals analyzed are indicated (n). 4-6 sections (A-C) or 3-4 sections (D) of each animal were examined. A, CA3; B, CA1; C, dentate gyrus. Student's t-test was performed for A-C. A significant difference (decrease by 18%) was revealed for CA3 (A,p=0.0318) but not for CA1 (B, p=0.0803) or dentate gyrus (C,p=0.1781). Analysis of variance (one-way ANOVA) was performed for data in D (p=0.5344). Significance (p<0.05) is indicated by an asterisk.

FIGS. 13A and 13B are photomicrographs showing microscopic distribution of BC1 RNA in the CA3 field of the hippocampus after AD induction. Asterisk indicates the area that was punctured by electrode implantation on the stimulated side. The radiatum/pyramidale ratio of BC1 expression was not altered following AD induction. Luc, stratum lucidum; Py, stratum pyramidale; Rad, stratum radiatum. Scale bar, 200 μm.

FIGS. 14A and B are photomicrographs produced in control experiments to ascertain that induction of ADs did not result in tissue damage. A, B, Presynaptic specializations were visualized in the CA3 region of a seizured animal by immunocyto-chemistry. B shows fluorescence signal (red) for synaptophysin in the stimulated hippocampus, A in the control hemisphere. Mossy fiber terminals are abundant in both stimulated and unstimulated hippocampus. C, Expression of Arc mRNA after kindling of the right hemisphere. Stimulation paradigms were similar to other kindling experiments used in this works but yielded in a more generalized and bilateral RNA induction in this case. Arc mRNA expression was induced in all hippocampal areas including those in the immediate vicinity of the electrode puncture (arrow). Luc, stratum lucidum; Py, stratum pyramidale; Rad, stratum radiatum. Scale bar, 250 μm (A,B), 1000 μm (C).

FIG. 15A is a gel showing results of translation of programming EMCV.GFP mRNA in the presence of 100 nM BC1 RNA, titrated in RRL with full-length eIF4A and/or PABP. Relative signal intensities of GFP protein bands were quantified by phosphor-imaging and are listed for each lane. FIG. 14B graphically depicts results from three experiments which showed that on average, that translation in the presence of 400 nM of both eIF4A and PABP was restored to 86.7% of uninhibited translation [one-way ANOVA,p<0.001; Scheffe's multiple comparison post hoc analysis (comparison with lane 2): ***p<0.001 for lanes 1 and 4].

### DETAILED DESCRIPTION OF THE INVENTION

In accordance with the present invention, BC1 and BC200 RNA have been identified as specific repressors of translation It had been previously shown that BC1 RNA is specifically and rapidly transported to dendrites (Muslimov et al. 1997), and that somatodendritic BC1 expression levels are subject to activity-dependent modulation (Muslimov et al. 1998). In accordance with the present invention, it has now been discovered that BC1 and BC200 RNA are both specific repressors of translational initiation both in cap-dependent and internal entry modes. In particular, these RNAs repress translation by inhibiting initiation at the level of 48S complex assembly. In accordance with the present invention, BC1-mediated repression has been shown to be effective not only in cap-dependent translation initiation but also in eIF4-dependent internal initiation. Thus, non-translatable BC1 and BC200 RNA play a functional role in translational control of local protein synthesis in nerve cells.

Expression of the small neuronal non-coding transcript BC200 RNA is tightly regulated. The RNA is not normally detected in non-neuronal somatic cells. As described in U.S. Pat. Nos. 5,670,318 and 5,736,329, the tight neuron-specific control of BC200 expression is deregulated in various tumors, including breast tumors. BC200 RNA is associated with malignancy and is not detectable in normal non-neuronal somatic tissue or in benign tumors such as fibroadenomas of the breast. Amounts of BC200 RNA expressed by cancerous tumor cells of the breast correlate with tumor type, grade and stage. BC200 RNA is expressed at high levels in invasive carcinomas.

BC200 expression levels are also drastically increased in several cortical areas of the brains of patients suffering from Alzheimer's disease. See e.g., U.S. Pat. Nos. 5,670,318 and 5,736,329. In accordance with the present invention, it has also been discovered that BC1 RNA is downregulated in response to the induction of epileptiform activity. Specifically, levels of BC1 RNA are significantly reduced ipsilaterally in CA3. The mechanistic basis for the reduction of BC1 RNA is not yet know, i.e., whether the reduction is due to decreased transcription and/or decreased degradation. It is likely that a downregulation of BC1 expression levels is a mechanism that promotes synaptodendritic protein synthesis, thereby facilitating epileptogenesis.

Based on these discoveries outlined above, the present invention provides methods for modulating the level of BC200 RNA present in neuronal domains such as somata and postsynaptic domains, as well as in cancerous tissue. In the case of cancer patients and Alzheiner's patients, downregulation of BC200 RNA may be performed using a variety of well known methods such as gene silencing, antisense and dominant/negative mutants. With respect to treating patients suffering from epilepsy, levels of BC200 RNA may be increased via administration of therapeutically effective amounts of BC200 RNA or via gene therapy. In the present context, a vector which can replicate within a subject comprises DNA or RNA corresponding to BC200 RNA, which DNA or RNA is operably linked to a promoter sequence which functions in a cell of a subject. For example, if the subject is a human, there are various promoters which may be used in order to drive expression of a BC200 RNA or DNA in a human cell. BC200 transcripts are therefore made available within a subject. By tailoring the promoter which drives expression of the BC200 DNA or RNA, neuronal specific, tumor specific and/or brain specific expression of BC200 may be achieved. For example, in order to express BC200 RNA in neurons, an NSE (neuron-specific enolase) or CaMKII alpha (calcium-calmodulin dependent protein kinase II alpha subunit) promoter may be used. Expression of BC200 RNA transcripts in neoplastic cells within a subject may be achieved using a cell type specific promoter(s). There is a wealth of information available on gene therapy which may be also be used in order to practice to present invention. Examples of useful references include, e.g., Sauter et al. 2003 and Hall, S. J., et al., (1997).

While antisense targeting is the preferred method for interfering with BC200 RNA transcript levels in order to down regulate BC200, other methods known to those skilled in the art can be used to interfere with BC200 RNA These include, but are not limited to, small interfering RNAs, which are sequence-specific reagents capable of suppressing the expression of genes through RNA interference. Such methods are described, for example, by Tuschl, Expanding small RNA interference, Nature Biotechnology, vol. 20, pp. 446-448 (May 2002), Miyagishi et al., U6 Promoter-Driven siRNAs With Four Uridine 3′ Overhangs Efficiently Suppress Targeted Gene Expression in Mammalian Cells, Nature Biotechnology, vol. 20, pp. 497-500 (May 2002); Lee et al., Expression of Small Interfering RNAs Targeted Against HIV-1 rev Transcripts in Human Cells, Nature Biotechnology, vol. 20, pp. 500-505 (May 2002); Paul et al., Effective Expression of Small Interfering RNA in Human Cells, Nature Biotechnology, vol. 20, pp. 505-508 (May 2002), the contents of each of which are incorporated by reference herein.

BC200 RNA may also be neutralized by the introduction of dominant negative mutants. The introduction of such mutants would have the consequence that endogenous BC200 RNA would face a mutant BC200 RNA that would compete with the endogenous RNA for certain binding sites, but would be functionally incompetent. For instance, Example 2 herein, demonstrates that rodent BC1 RNA interacts simultaneously with eukaryotic initiation factor 4A (eIF4A) and poly(A) binding protein (PABP). Further, Example 5 herein, shows that this simultaneous interaction is necessary for translational repression. Interaction with only one of the two factors is not sufficient for repression. Thus, one could engineer a mutant that would bind to only one factor. Through this binding, it would block that factor from interacting with endogenous BC1 RNA but, since unable to interact with the other factor, would not be functionally active. Since PABP binds to A-rich elements in the central and 3′ part of BC1 RNA and BC200 RNA, one could mutate these elements to random sequence or U-rich elements to generate mutants that would still bind eIF4A but not PABP.

With respect to down regulation of BC200 via antisense technology, the present invention utilizes oligonucleotides targeted to BC200, i.e., antisense molecules, as a means for treating both neurological disorders and carcinomas. The target of the antisense technology is BC200 RNA, a non-translated RNA marker associated with malignancy and certain neurological disorders, including Alzheimer's Disease, that is not detectable in normal non-neuronal somatic tissue or in benign tumors such as fibroadenomas of the breast. Suitable oligonucleotides for use as antisense oligonucleotides include the probes described above in U.S. Pat. Nos. 5,670,318 and 5,736,329.

The present invention employs oligomeric compounds, particularly antisense oligonucleotides, for use in modulating the function of nucleic acid molecules encoding BC200 RNA This is accomplished by providing antisense compounds which specifically hybridize with one or more nucleic acids encoding BC200 RNA As used herein, the terms “target nucleic acid” and “nucleic acid encoding BC200 RNA” encompass DNA encoding BC200 RNA, RNA (including pre-mRNA and mRNA) transcribed from such DNA, including BC200 RNA itself, and also cDNA derived from such RNA. The specific hybridization of an oligomeric compound with its target nucleic acid interferes with the normal function of the nucleic acid. This modulation of function of a target nucleic acid by compounds which specifically hybridize to it is generally referred to as “antisense”.

The functions of DNA to be interfered with include replication and transcription. The functions of RNA to be interfered with include all vital functions such as, for example, translocation of the RNA to the site of protein translation, translation of protein from the RNA, splicing of the RNA to yield one or more mRNA species, and catalytic or other (e.g. inhibitory) activity which may be engaged in or facilitated by the RNA. The overall effect of such interference with target nucleic acid function is modulation of the expression of BC200 RNA. In the context of the present invention, “modulation” means either an increase (stimulation) or a decrease (inhibition) in the expression of a gene. In the context of the present invention, inhibition is the preferred form of modulation of gene expression, and BC200 RNA is a preferred target.

As noted in U.S. Pat. No. 5,736,329, BC200 RNA is a 200-nucleotide long non-translatable RNA, having the following primary sequence:

(SEQ ID NO 1)

XXCCGGGCGC GGUGGCUCAC GCCUGUAAUC CCAGCUCUCA
GGGAGGCUAA GAGGCGGGAG GAUAGCUUGA GCCCAGGAGU
UCGAGACCUG CCUGGGCAAU AUAGCGAGAC CCCGUUCUCC
AGAAAAAGGA AAAAAAAAAA CAAAAGACAA AAAAAAAAUA
AGCGUAACUU CCCUCAAAGC AACAACCCCC CCCCCCCUUU

The X's at positions 1 and 2 are independently either G or absent.

Preferably, the antisense compounds of this invention are targeted to a specific portion of BC200 RNA identified above in SEQ ID NO:1 so that they inhibit the function of BC200 RNA.

More preferably, the antisense compounds used to inhibit BC200 RNA in a sample are oligonucleotides which are complementary to the unique sequences of Domain III of human BC200 RNA, or to corresponding chromosomal DNA, i.e., which are complementary to at least a portion of the sequence:

(SEQ ID NO 2)

UAAGCGUAAC UUCCCUCAAA GCAACAACCC CCCCCCCCCU UU

Such antisense compounds are linear oligonucleotides containing from about 10 to 60 bases. The length must be sufficient to provide a reasonable degree of specificity such that binding with BC200 RNA will be preferred over binding to other polynucleotides.

One antisense compound witliin the scope of the invention is complementary to the nucleotides 156-185 of BC200 RNA. This 30-nucleotide antisense compound has the sequence:

(SEQ ID NO 3)

TTGTTGCTTT GAGGGAAGTT ACGCTTATTT

As one skilled in the art would recognize, the “T” (thymine) of the above sequence (or any sequence herein) would be replaced with “U” (uracil) where the antisense compound is RNA.

Another useful antisense compound is a 21-nucleotide probe complementary to nucleotides 158-178, i.e.:

(SEQ ID NO 4)

TTTGAGGGAA GTTACGCTTA T

Suitable antisense compounds may be complementary with the portions of BC200 RNA outside Domain III. Preferably, the antisense compounds are also complementary to a portion (i.e., at least about 10 bases) of the unique Domain III sufficient to provide specificity. Antisense compounds may also be complementary to portions of Domain III alone. A further aspect of the invention is a second class of antisense compounds which are complementary to a portion of Domain II spanning nucleotides 146-148.

In a still further aspect of the invention, antisense compounds can be utilized which are complementary to and specifically hybridize with a portion of the Alu-repetitive sequence spanning the two unique nucleotides at positions 48 and 49 of BC200 RNA or corresponding DNA. Examples of such antisense compounds are:

(SEQ ID NO 5)

CCTCTTAGCC TCCCTGAGAG CT

a particularly useful antisense compound that will bind BC200 RNA and:

(SEQ ID NO 6)

CCAGCTCTCA GGGAGGCTAA

a sense compound that will bind to corresponding DNA sequences. These antisense compounds can be used for detection or inhibition of BC200 RNA.

Modifications to the antisense molecules set forth in SEQ ID NOs:3-6, which modifications do not effect the ability of the oligonucleotide to bind to BC200, are also within the scope of the present invention. Such modifications include insertions, deletions and substitutions of one or more nucleotides.

It is preferred to target specific nucleic acids for antisense. “Targeting” an antisense compound to a particular nucleic acid, in the context of this invention, is a multistep process. The process usually begins with the identification of a nucleic acid sequence whose function is to be modulated. This may be, for example, a cellular gene (or RNA transcribed from the gene) whose expression is associated with a particular disorder or disease state, or a nucleic acid molecule from an infectious agent. In the present invention, the target is a nucleic acid molecule encoding BC200 RNA, most preferably BC200 RNA itself. The targeting process also includes determination of a site or sites within this nucleic acid for the antisense interaction to occur such that the desired effect, e.g., detection or modulation of expression of the protein, will result.

Once one or more target sites have been identified, oligonucleotides are chosen which are sufficiently complementary to the target, i.e., hybridize sufficiently well and with sufficient specificity, to give the desired effect.

In the context of this invention, “hybridization” means hydrogen bonding, which may be Watson-Crick, Hoogsteen or reversed Hoogsteen hydrogen bonding, between complementary nucleoside or nucleotide bases. For example, adenine and thymine are complementary nucleobases which pair through the formation of hydrogen bonds. “Complementary,” as used herein, refers to the capacity for precise pairing between two nucleotides. For example, if a nucleotide at a certain position of an oligonucleotide is capable of hydrogen bonding with a nucleotide at the same position of a DNA or RNA molecule, then the oligonucleotide and the DNA or RNA are considered to be complementary to each other at that position. The oligonucleotide and the DNA or RNA are complementary to each other when a sufficient number of corresponding positions in each molecule are occupied by nucleotides which can hydrogen bond with each other. Thus, “specifically hybridizable” and “complementary” are terms which are used to indicate a sufficient degree of complementarity or precise pairing such that stable and specific binding occurs between the oligonucleotide and the DNA or RNA target. It is understood-in the art that the sequence of an antisense compound need not be 100% complementary to that of its target nucleic acid to be specifically hybridizable. An antisense compound is specifically hybridizable when binding of the compound to the target DNA or RNA molecule interferes with the normal function of the target DNA or RNA to cause a loss of utility, and there is a sufficient degree of complementarity to avoid non-specific binding of the antisense compound to non-target sequences under conditions in which specific binding is desired, i.e., under physiological conditions in the case of in vivo assays or therapeutic treatment, and in the case of in vitro assays, under conditions in which the assays are performed.

Antisense and other compounds of the invention which hybridize to the target and inhibit expression of the target are identified through experimentation, and the sequences of these compounds are preferred embodiments ofthe invention. The target sites to which these preferred sequences are complementary are “active sites” and are therefore preferred sites for targeting. Therefore another embodiment of the invention encompasses compounds which hybridize to these active sites.

Expression patterns within cells or tissues treated with one or more antisense compounds are compared to control cells or tissues not treated with antisense compounds and the patterns produced are analyzed for differential levels of gene expression as they pertain, for example, to disease association, signaling pathway, cellular localization, expression level, size, structure or function of the genes examined. These analyses can be performed on stimulated or unstimulated cells and in the presence or absence of other compounds which affect expression patterns.

Examples of methods of gene expression analysis known in the art include DNA arrays or microarrays (Brazma and Vilo, FEBS Lett., 2000, 480, 17-24; Celis, et al., FEBS Lett., 2000, 480, 2-16), SAGE (serial analysis of gene expression) (Madden, et al., Drug Discov. Today, 2000, 5, 415-425), READS (restriction enzyme amplification of digested cDNAs) (Prashar and Weissman, Methods Enzymol., 1999, 303, 258-72), TOGA (total gene expression analysis) (Sutcliffe, et al., Proc. Natl. Acad. Sci. U.S.A., 2000, 97, 1976-81), protein arrays and proteomics (Celis, et al., FEBS Lett., 2000, 480, 2-16; Jungblut, et al., Electrophoresis, 1999, 20, 2100-10), expressed sequence tag (EST) sequencing (Celis, et al., FEBS Lett., 2000, 480, 2-16; Larsson, et al., J. Biotechnol., 2000, 80, 143-57), subtractive RNA fingerprinting (SuRF) (Fuchs, et al., Anal. Biochem., 2000, 286, 91-98; Larson, et al., Cytometry, 2000, 41, 203-208), subtractive cloning, differential display (DD) (Jurecic and Belmont, Curr. Opin. Microbiol., 2000, 3, 316-21), comparative genomic hybridization (Carulli, et al., J. Cell Biochem. Suppl., 1998, 31, 286-96), FISH (fluorescent in situ hybridization) techniques (Going and Gusterson, Eur. J. Cancer, 1999, 35, 1895-904) and mass spectrometry methods (reviewed in (To, Comb. Chem. High Throughput Screen, 2000, 3, 235-41).

In the context of this invention, the term “oligonucleotide” refers to an oligomer or polymer of ribonucleic acid (RNA) or deoxyribonucleic acid (DNA) or mimetics thereof. This term includes oligonucleotides composed of naturally-occurring nucleobases, sugars and covalent intemucleoside (backbone) linkages as well as oligonucleotides having non-naturally-occurring portions which function similarly. Such modified or substituted oligonucleotides are often preferred over native forms because of desirable properties such as, for example, enhanced cellular uptake, enhanced affinity for nucleic acid target and increased stability in the presence of nucleases.

While antisense oligonucleotides are a preferred form of antisense compound, the present invention comprehends other oligomeric antisense compounds, including but not limited to oligonucleotide mimetics. The antisense compounds in accordance with this invention preferably comprise from about 8 to about 50 nucleobases (i.e. from about 8 to about 50 linked nucleosides). Particularly preferred antisense compounds are antisense oligonucleotides, even more preferably those comprising from about 12 to about 30 nucleobases. Antisense compounds include ribozymes, external guide sequence (EGS) oligonucleotides (oligozymes), and other short catalytic RNAs or catalytic oligonucleotides which hybridize to the target nucleic acid and modulate its expression.

As is known in the art a nucleoside is a base-sugar combination. The base portion of the nucleoside is normally a heterocyclic base. The two most common classes of such heterocyclic bases are the purines and the pyrimidines. Nucleotides are nucleosides that firther include a phosphate group covalently linked to the sugar portion of the nucleoside. For those nucleosides that include a pentofuranosyl sugar, the phosphate group can be linked to either the 2′, 3′ or 5′ hydroxyl moiety of the sugar. In forming oligonucleotides, the phosphate groups covalently link adjacent nucleosides to one another to form a linear polymeric compound. In turn the respective ends of this linear polymeric structure can be further joined to form a circular structure, however, open linear structures are generally preferred. Within the oligonucleotide structure, the phosphate groups are commonly referred to as forming the internucleoside backbone of the oligonucleotide. The normal linkage or backbone of RNA and DNA is a 3′ to 5′ phosphodiester linkage.

Other antisense compounds include oligonucleotides containing modified backbones or non-natural intemucleoside linkages. As used herein, oligonucleotides having modified backbones include those that retain a phosphorus atom in the backbone and those that do not have a phosphorus atom in the backbone. For the purposes herein, and as sometimes referenced in the art, modified oligonucleotides that do not have a phosphorus atom in their intemucleoside backbone can also be considered to be oligonucleosides.

Modified oligonucleotide backbones include, for example, phosphorothioates, chiral phosphorothioates, phosphorodithioates, phosphotriesters, aminoalkylphosphotriesters, methyl and other alkyl phosphonates including 3′-alkylene phosphonates, 5′-alkylene phosphonates and chiral phosphonates, phosphinates, phosphoramidates including 3′-amino phosphoramidate and aminoalkylphosphoramidates, thionophosphorarnidates, thionoalkylphosphonates, thionoalkylphosphotriesters, selenophosphates and boranophosphates having normal 3′-5′ linkages, 2′-5′ linked analogs of these, and those having inverted polarity wherein one or more intemucleotide linkages is a 3′ to 3′, 5′ to 5′ or 2′ to 2′ linkage. Representative United States patents that teach the preparation of the above phosphorus-containing linkages include, but are not limited to, U.S. Pat. Nos.: 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; 5,194,599; 5,565,555; 5,527,899; 5,721,218; 5,672,697 and 5,625,050, each of which is herein incorporated by reference.

Modified oligonucleotide backbones that do not include a phosphorus atom therein can have backbones that are formed-by short chain alkyl or cycloalkyl intemucleoside linkages, mixed heteroatom and alkyl or cycloalkyl intemucleoside linkages, or one or more short chain heteroatomic or heterocyclic intemucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CH₂ component parts.

Representative United States patents that teach the preparation of the above oligonucleosides include, but are not limited to, U.S. Pat. Nos.: 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; 5,792,608; 5,646,269 and 5,677,439, each of which are herein incorporated by reference.

In other oligonucleotide mimetics, both the sugar and the intemucleoside linkage, i.e., the backbone, of the nucleotide units are replaced with novel groups. The base units are maintained for hybridization with an appropriate nucleic acid target compound. One such oligomeric compound, an oligonucleotide mimetic that has been shown to have-excellent hybridization properties, is referred to as a peptide nucleic acid (PNA). In PNA compounds, the sugar-backbone of an oligonucleotide is replaced with an amide containing backbone, in particular an aminoethylglycine backbone. The nucleobases are retained and are bound directly or indirectly to aza nitrogen atoms of the amide portion of the backbone. Representative United States patents that teach the preparation of PNA compounds include, but are not limited to, U.S. Pat. Nos.: 5,539,082; 5,714,331; and 5,719,262, each of which is herein incorporated by reference. Further teaching of PNA compounds can be found in Nielsen et al., Science, 1991, 254, 1497-1500.

Suitable oligonucleotides for use in the present invention are those possessing phosphorothioate backbones and suitable oligonucleosides are those possessing heteroatom backbones, and in particular —CH₂—NH—O—CH₂—, —CH₂—N(CH₃)—O—CH— [known as a methylene (methylimino) or MMI backbone], —CH₂—O—N(CH₃)—CH₂—, —CH₂—N(CH₃)—N(CH₃)—CH₂— and —O—N(CH₃)—CH₂—CH₂— [wherein the native phosphodiester backbone is represented as —O—P—O—CH₂—] of the above referenced U.S. Pat. No. 5,489,677, and the amide backbones of the above referenced U.S. Pat. No. 5,602,240. Also suitable are oligonucleotides having morpholino backbone structures of the above-referenced U.S. Pat No. 5,034,506.

Modified oligonucleotides may also contain one or more substituted sugar moieties. Such oligonucleotides may comprise one of the following at the 2′ position: OH; F; O—, S—, or N-alkyl; O—, S—, or N-alkenyl; O—, S—or N-alkynyl; or O-alkyl-O-alkyl, wherein the alkyl, alkenyl and alkynyl may be substituted or unsubstituted C₁ to C₁₀ alkyl or C₂ to C₁₀ alkenyl and alkynyl. Particularly preferred are O[(CH₂)ₙO]ₘ CH₃, O(CH₂)ₙ OCH₃, O(CH₂)ₙ NH₂, O(CH₂)ₙ CH₃, O(CH₂)ₙ ONH₂, and O(CH₂)ₙ ON[(CH₂)ₙ CH₃)]₂, where n and m are from 1 to about 10. Other oligonucleotides comprise one of the following at the 2′ position: C₁ to C₁₀ lower alkyl, substituted lower alkyl, alkenyl, alkynyl, alkaryl, aralkyl, O-alkaryl or O-aralkyl, SH, SCH₃, OCN, Cl, Br, CN, CF, OCF, SOCH₃, SO₂ CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. Other modifications include 2′-methoxyethoxy (2′-O—CH₂ CH₂ OCH₃, also known as 2′-O—(2-methoxyethyl) or 2′-MOE) (Martin et al., Helv. Chim. Acta, 1995, 78, 486-504) i.e., an alkloxyalkoxy group. A further modification may include 2′-dimethylaminooxyethoxy, i.e., a O(CH₂) ₂ ON(CH₃)₂ group, also known as 2′-DMAOE, and 2′-dimethylaminoethoxyethoxy (also known in the art as 2′-O-dimethylaminoethoxyethyl or 2′-DMAEOE), i.e., 2′-O—CH₂—O—CH₂—N(CH₂)₂.

Other preferable modifications include Locked Nucleic Acids (LNAs) in which the 2′-hydroxyl group is linked to the 3′ or 4′ carbon atom of the sugar ring thereby forming a bicyclic sugar moiety. The linkage is preferably a methelyne (—CH₂—)ₙ group bridging the 2′ oxygen atom and the 4′ carbon atom wherein n is 1 or 2. LNAs and preparation thereof are described in WO 98/39352 and WO 99/14226, the contents of each of which are incorporated by reference herein.

Other preferred modifications include 2′-methoxy (2′-O—CH₃), 2′-aminopropoxy (2′-OCH₂CH₂ CH₂ NH₂), 2′-allyl (2′-CH₂—CH═CH₂), 2′-O-allyl (2′—O—CH₂—CH═CH₂) and 2′-fluoro (2′-F). The 2′-modification may be in the arabino (up) position or ribo (down) position. A preferred 2′-arabino modification is 2′-F. Similar modifications may also be made at other positions on the oligonucleotide, particularly the 3′ position of the sugar on the 3′ terminal nucleotide or in 2′-5′ linked oligonucleotides and the 5′0 position of 5′ terminal nucleotide. Oligonucleotides may also have sugar mimetics such as cyclobutyl moieties in place of the pentofuranosyl sugar. Representative United States patents that teach the preparation of such modified sugar structures include, but are not limited to, U.S. Pat. Nos.: 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; 5,792,747; and 5,700,920, each of which are herein incorporated by reference in its entirety.

Oligonucleotides may also include nucleobase (often referred to in the art simply as “base”) modifications or substitutions. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl (—C≡C—CH₃) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine. Further modified nucleobases include tricyclic pyrimidines such as phenoxazine cytidine(1H-pyrimido[5,4-b][1,4]benzoxazin-2(3H)-one), phenothiazine cytidine (1H-pyrimido[5,4-b][1,4]benzothiazin-2(3H)-one), G-clamps such as a substituted phenoxazine cytidine (e.g. 9-(2-aminoethoxy)-H-pyrimido[5,4-b][1,4]benzoxazin-2(3H)-one), carbazole cytidine (2H-pyrimido[4,5-b]indol-2-one), pyridoindole cytidine (H-pyrido[3′,2′:4,5]pyrrolo[2,3-d]pyrirnidin-2-one). Modified nucleobases may also include those in which the purine or pyrimidine base is replaced with other heterocycles, for example 7-deaza-adenine, 7-deazaguanosine, 2-aminopyridine and 2-pyridone. Further nucleobases include those disclosed in U.S. Pat. No. 3,687,808, those disclosed in The Concise Encyclopedia Of Polymer Science And Engineering, pages 858-859, Kroschwitz, J. I., ed. John Wiley & Sons, 1990, those disclosed by Englisch et al., Angewandte Chemie, International Edition, 1991, 30, 613, and those disclosed by Sanghvi, Y. S., Chapter 15, Antisense Research and Applications, pages 289-302, Crooke, S. T. and Lebleu, B., ed., CRC Press, 1993. Particularly useful nucleobases for increasing the binding affinity of the oligomeric compounds include 5-substituted pyrimidines, 6-azapyrimidines and N-2, N-6 and O-6 substituted purines, including 2-aminopropyladenine, 5-propynyluracil and 5-propynylcytosine. 5-methylcytosine substitutions have been shown to increase nucleic acid duplex stability by 0.6-1.2.degree. C. (Sanghvi, Y. S., Crooke, S. T. and Lebleu, B., eds., Antisense Research and Applications, CRC Press, Boca Raton, 1993, pp. 276-278) and are presently preferred base substitutions, even more particularly when combined with 2′-O-methoxyethyl sugar modifications.

Representative United States patents that teach the preparation of certain of the above noted modified nucleobases as well as other modified nucleobases include, but are not limited to, the above noted U.S. Pat. No. 3,687,808, as well as U.S. Pat Nos.: 4,845,205; 5,130,302; 5,134,066; 5,175,273; 5,367,066; 5,432,272; 5,457,187; 5,459,255; 5,484,908; 30 5,502,177; 5,525,711; 5,552,540; 5,587,469; 5,594,121, 5,596,091; 5,614,617; 5,645,985; 5,830,653; 5,750,692; 5,763,588; 6,005,096; and 5,681,941, each of which are herein incorporated by reference.

Other modifications of the oligonucleotides of the invention can involve chemically linking to the oligonucleotide one or more moieties or conjugates which enhance the activity, cellular distribution or cellular uptake of the oligonucleotide. The compounds of the invention can include conjugate groups covalently bound to functional groups such as primary or secondary hydroxyl groups. Conjugate groups of the invention include intercalators, reporter molecules, polyamines, polyamides, polyethylene glycols, polyethers, groups that enhance the pharmacodynamic properties of oligomers, and groups that enhance the pharmacokinetic properties of oligomers. Typical conjugates groups include cholesterols, lipids, phospholipids, biotin, phenazine, folate, phenanthridine, anthraquinone, acridine, fluoresceins, rhodamines, coumarins, and dyes. Groups that enhance the pharmacodynamic properties, in the context of this invention, include groups that improve oligomer uptake, enhance oligomer resistance to degradation, and/or strengthen sequence-specific hybridization with RNA. Groups that enhance the pharmacokinetic properties, in the context of this invention, include groups that improve oligomer uptake, distribution, metabolism or excretion. Conjugate moieties include but are not limited to lipid moieties such as a cholesterol moiety (Letsinger et al., Proc. Natl. Acad. Sci. USA, 1989, 86, 6553-6556), cholic acid (Manoharan et al., Bioorg. Med. Chem. Let., 1994, 4, 1053-1060), a thioether, e.g., hexyl-S-tritylthiol (Manoharan et al., Ann N.Y. Acad. Sci., 1992, 660, 306-309; Manoharan et al., Bioorg. Med. Chem. Let., 1993, 3, 2765-2770), a thiocholesterol (Oberhauser et al., Nucl. Acids Res., 1992, 20, 533-538), an aliphatic chain, e.g., dodecandiol or undecyl residues (Saison-Behmoaras et al., EMBO J., 1991, 10, 1111-1118; Kabanov et al., FEBS Lett., 1990, 259, 327-330; Svinarchuk et al., Biochimie, 1993, 75, 49-54), a phospholipid, e.g., di-hexadecyl-rac-glycerol or triethylammonium 1,2-di-O-hexadecyl-rac-glycero-3-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Manoharan et al., Nucleosides & Nucleotides, 1995, 14,969-973), or adamantane acetic acid (Manoharan et al., Tetrahedron Lett., 1995, 36, 3651-3654), a palmityl moiety (Mishra et al., Biochim. Biophys. Acta, 1995, 1264, 229-237), or an octadecylamine or hexylamino-carbonyl-oxycholesterol moiety (Crooke et al., J. Pharmacol. Exp. Ther., 1996, 277, 923-937.

Oligonucleotides of the invention may also be conjugated to active drug substances, for example, aspirin, warfarin, phenylbutazone, ibuprofen, suprofen, fenbufen, ketoprofen, (S)-(+)-pranoprofen, carprofen, dansylsarcosine, 2,3,5-triiodobenzoic acid, flufenamic acid, folinic acid, a benzothiadiazide, chlorothiazide, a diazepine, indomethicin, a barbiturate, a cephalosporin, a sulfa drug, an antidiabetic, an antibacterial or an antibiotic.

Representative United States patents that teach the preparation of such oligonucleotide conjugates include, but are not limited to, U.S. Pat. Nos.: 4,828,979; 4,948,882; 5,218,105; 5,525,465; 5,541,313; 5,545,730; 5,552,538; 5,578,717, 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941, each of which are herein incorporated by reference.

Antisense compounds useful in accordance with the present disclosure can be labeled. A variety of enzymes can be used to attach radiolabels (using dNTP precursors) to DNA termini. The 3′ termini of double stranded DNA can for example be labeled by using the Klenow fragment of E. coli DNA polymerase I. Blunt ended DNA or recessed 3′ termini are appropriate substrates. T4 DNA polymerase can also be used to label protruding 3′ ends. T4 polynucleotide kinase can be used to transfer a ³²P-phosphate group to the 5′ termini of DNA. This reaction is particularly useful to label single stranded oligonucleotides. Probes can also be labeled via PCR labeling in which labeled nucleic acids and/or labeled primers are used in PCR generation of probes from an appropriate clone. See Kelly et al., Genomics 13: 381-388 (1992).

The present invention also includes antisense compounds which are chimeric compounds. “Chimeric” antisense compounds or “chimeras,” in the context of this invention, are antisense compounds, particularly oligonucleotides, which contain two or more chemically distinct regions, each made up of at least one monomer unit, i.e., a nucleotide in the case of an oligonucleotide compound. These oligonucleotides typically contain at least one region wherein the oligonucleotide is modified so as to confer upon the oligonucleotide increased resistance to nuclease degradation, increased cellular uptake, and/or increased binding affinity for the target nucleic acid. An additional region of the oligonucleotide may serve as a substrate for enzymes capable of cleaving RNA:DNA or RNA:RNA hybrids. By way of example, RNase H is a cellular endonuclease which cleaves the RNA strand of an RNA:DNA duplex. Activation of RNase H, therefore, results in cleavage of the RNA target, thereby. greatly enhancing the efficiency of oligonucleotide inhibition of gene expression. Consequently, comparable results can often be obtained with shorter oligonucleotides when chimeric oligonucleotides are used, compared to phosphorothioate deoxyoligonucleotides hybridizing to the same target region. Cleavage of the RNA target can be routinely detected by gel electrophoresis and, if necessary, associated nucleic acid hybridization techniques known in the art.

Chimeric antisense compounds of the invention may be formed as composite structures of two or more oligonucleotides, modified oligonucleotides, oligonucleosides and/or oligonucleotide mimetics as described above. Such compounds have also been referred to in the art as hybrids or gapmers. Representative United States patents that teach the preparation of such hybrid structures include, but are not limited to, U.S. Pat. Nos.: 5,013,830; 5,149,797; 5,220,007; 5,256,775; 5,366,878; 5,403,711; 5,491,133; 5,565,350; 5,623,065; 5,652,355; 5,652,356; and 5,700,922, each of which are herein incorporated by reference.

The compounds of the invention may also be admixed, encapsulated, conjugated or otherwise associated with other molecules, molecule structures or mixtures of compounds, as for example, liposomes, receptor targeted molecules, oral, rectal, topical or other formulations, for assisting in uptake, distribution and/or absorption. Representative United States patents that teach the preparation of such uptake, distribution and/or absorption assisting formulations include, but are not limited to, U.S. Pat. Nos.: 5,108,921; 5,354,844; 5,416,016; 5,459,127; 5,521,291; 5,543,158; 5,547,932; 5,583,020; 5,591,721; 4,426,330; 4,534,899; 5,013,556; 5,108,921; 5,213,804; 5,227,170; 5,264,221; 5,356,633; 5,395,619; 5,416,016; 5,417,978; 5,462,854; 5,469,854; 5,512,295; 5,527,528; 5,534,259; 5,543,152; 5,556,948; 5,580,575; and 5,595,756, each of which are herein incorporated by reference.

The antisense compounds of the invention also encompass any pharmaceutically acceptable salts, esters, or salts of such esters, or any other compound which, upon administration to an animal including a human, is capable of providing (directly or indirectly) the biologically active metabolite or residue thereof Accordingly, the disclosure is also drawn to prodrugs and pharmaceutically acceptable salts of the compounds of the invention, pharmaceutically acceptable salts of such prodrugs, and other bioequivalents.

The antisense compounds utilized in accordance with the present disclosure can be made by any of a variety of techniques known in the art. They may be conveniently and routinely made through the well-known technique of solid phase synthesis. Equipment for such synthesis is sold by several vendors including, for example, Applied Biosystems (Foster City, Calif.). Any other means for such synthesis known in the art may additionally or alternatively be employed. It is well known to use similar techniques to prepare oligonucleotides such as the phosphorothioates and alkylated derivatives. For example, cyanoethyl phosphoramidite chemistry may be used to produce phosphorothioate oligonucleotides.

In addition, antisense compounds can be generated by in vitro transcription In this approach, the desired sequence is first cloned into a suitable transcription vector (e.g., pBluescript). This vector is linearized so that transcription will terminate at a specific location, and RNA is transcribed from such linearized templates, using SP6, T3, or T7 RNA polymerase. The antisense compounds can be ³⁵S- or ³H-labeled by adding the appropriate radiolabeled precursors to the reaction mixture. Template DNA is then digested with DNase I. RNA antisense compounds can be further purified by gel filtration or gel electrophoresis.

Antisense compounds can also be made by oligolabeling, although this technique is more suited to longer nucleic acid polymers. In this method, double stranded DNA is first denatured. Random sequence oligonucleotides are then used as primers for the template directed synthesis of DNA. The Klenow fragment of E. coli DNA polymerase I is frequently used in this application. Reverse transcriptase can be used if the template is RNA. Labeling of the antisense compounds is achieved by incorporation of radiolabeled nucleotides.

Single stranded DNA antisense compounds can be made from templates derived from bacteriophage M13 or similar vectors. An oligonucleotide primer, complementary to a specific segment of the template, is then used with the Klenow fragment of E. coli DNA polymerase I to generate a radiolabeled strand complementary to the template. The antisense compound is purified for example by gel electrophoresis under denaturing conditions.

Oligonucleotides of any desired sequence can also be synthesized chemically. As noted above, solid phase methods are routinely used in the automated synthesis of oligonucleotides.

The antisense compounds of the present invention can be utilized for diagnostics, therapeutics, prophylaxis and as research reagents and kits. For therapeutics, an animal, preferably a human, suspected of having a disease or disorder which can be treated by modulating the expression of BC200 RNA is treated by administering an antisense compound or BC200 RNA in accordance with this invention. The compounds of the invention can be utilized in pharmaceutical compositions by adding an effective amount of an antisense compound or BC200 RNA to a suitable pharmaceutically acceptable diluent or carrier. Use of the antisense compounds and BC200 RNA, and methods of the invention may also be useful prophylactically, e.g., to prevent or delay disease onset, inflammation or tumor formation, for example. As herein, “subject” or “patient” can encompass any animal, preferably a mammal, even more preferably, a human.

The present invention also includes pharmaceutical compositions and formulations which comprise the subject antisense oligonucleotides or BC200 RNA of the present invention and a pharmaceutically acceptable carrier. Dosages may be readily determined by one of ordinary skill in the art based on preferred effective amounts and formulated into the subject pharmaceutical compositions.

As used herein, “pharmaceutically acceptable carrier” includes any and all solvents, buffers, dispersion media, coatings, antibacterial and antifgal agents, isotonic and absorption delaying agents, and the like that are non-toxic to a subject The use of such media and agents for pharmaceutical active substances is well known in the arL Except insofar as any conventional media or agent is incompatible with the subject oligonucleotides or BC200 RNA, its use in the pharmaceutical compositions is contemplated. Supplementary active ingredients may also be incorporated into the compositions.

The pharmaceutical compositions of the present invention may be administered in a number of ways depending upon whether local or systemic treatment is desired and upon the area to be treated. Administration may be topical (including ophthalmic and to mucous membranes including vaginal and rectal delivery), pulmonary, e.g., by inhalation or insufflation of powders or aerosols, including by nebulizer; intratracheal, intranasal, epidermal and transdermal), oral or parenteral. Parenteral administration includes intravenous, intraarterial, subcutaneous, intraperitoneal or intramuscular injection or infusion; or intracranial, e.g., intrathecal or intraventricular, administration.

As set forth in detail below in the Examples, both BC1 and BC200 RNA are specific repressors of translation in dendrites. Accordingly, elevated levels of BC200 RNA has a role in the development of neurological disorders. Moreover, elevated levels of BC200 RNA has been found in carcinomas. Therefore, in a preferred embodiment, the antisense compounds of the present invention are utilized as therapeutics to treat disorders characterized by an increase in levels of BC200 RNA. More preferably, the antisense compounds are utilized to treat carcinomas and neurological disorders including, but not limited to, Alzheimer's Disease, Fragile X Mental Retardation Syndrome, Down's Syndrome and Parkinson's Disease.

Phosphorothiolate oligonucleotides are enzymatically stable and have been shown to be absorbed orally. Moreover, phosphorothiolate oligonucleotides can be delivered to the brain in effective doses by intravenous administration. Agrawal et al. (1995)

The dose of a subject antisense oligonucleotide or BC200 RNA to be administered to a subject in the context of the present invention, should be sufficient to effect a beneficial therapeutic response in the subject over time, and/or to alleviate symptoms. Thus, in accordance with the present invention, a subject antisense oligonucleotide or BC200 is administered to a patient in an amount sufficient to alleviate, reduce, ameliorate, cure or at least partially arrest symptoms and/or complications from the disease. An amount adequate to accomplish at least one of these effects is defined as a “therapeutically effective amount” or a “therapeutically effective dose.”

A therapeutically effective amount of a subject oligonucleotide and/or BC200 RNA will vary from patient to patient and is largely empirical. Considerations based on age, weight, type of disorder to be treated, e.g., neuronal disorder vs. cancer, type of cancer, and stage of disease may all be considered. It may be generally stated that a suitable dosage range is one which provides up to about 1 mu.g. to about 1,000 mu.g. to about 5,000 mu.g. to about 10,2000 mu.g. to about 25,000 mu.g or about 50,000 mu.g. of oligonucleotide per ml of carrier in a single dosage. Preferably, dosage is from 0.01 mu.g. to 100 g per kg of body weight, and may be given once or more daily, weekly, monthly, yearly, or even on a less frequent basis dependent on the needs of the patient. Optimal dosing schedules may be calculated from measurements of drug accumulation in a body of a patient.

In another aspect of the invention, there is provided a method of treating a neurological disorder such as Alzheimer's disease or cancer in a subject The method comprises down-regulating BC200 RNA transcript levels in a patient. For example, BC200 RNA transcript level may be down-regulated via administering a dominant negative mutant of BC200 RNA or a small interfering RNA at the dosages described above. BC200 RNA transcript levels may also be down-regulated by administering to a subject suffering from such disorder and/or in need of such treatment, a therapeutically effective amount of an antisense molecule targeted to the nucleotide sequence set forth in at least one of SEQ ID NO:1 or SEQ ID NO:2.

Alternatively, a method of treating a neurological disorder such as Alzheimer's disease or cancer in a subject comprises the steps of administering to a subject suffering from such disorder and/or in need of such treatment, a therapeutically effective amount of an antisense molecule comprising the nucleotide sequence set forth in SEQ IDNO:3. Such an antisense molecule is complementary to nucleotides 156-185 of BC200 RNA.

In still another embodiment of the invention, a method for treating a neurological disorder such as Alzheimer's disease or cancer comprises the steps of administering to a subject suffering from such disorder and/or in need of such treatment, a therapeutically effective amount of an antisense molecule comprising the nucleotide sequence set forth in SEQ IDNO:4. Such an antisense molecule is complementary to nucleotides 158-178 of BC200 RNA.

In a further embodiment, a method for treating a neurological disorder such as Alzheimer's disease or cancer in a subject comprises the steps of administering to a subject suffering from such disorder and/or in need of such treatment, a therapeutically effective amount of an antisense molecule comprising the nucleotide sequence set forth in SEQ ID NO:5

In a still further embodiment, a method for treating a neurological disorder such as Alzheimer's disease or cancer comprises the steps of administering to a subject suffering from such disorder and/or in need of such treatment, a therapeutically effective amount of an antisense molecule comprising the nucleotide sequence set forth in SEQ ID NO:6.

There are various types of neurological disorders which may be treated by the methods described above such as e.g., Alzheimer's disease, Fragile X mental retardation syndrome, Down's syndrome and Parkinson's disease.

There are various types of cancers which may also be treated via the methods described above. Examples include but are not limited to squamous cell carcinoma of the tongue and lung, epithelial carcinoma of the esophagus, tubular adenocarcinoma of the stomach, breast adenocarcinoma, adeno carcinoma of the lung, mucoepidermoid ofthe partoid gland, melanoma of the skin, papillary carcinoma of the ovaries, and endothelial adenocarcinoma of the cervix.

The present invention also provides a method for treating epilepsy in a patient. The method comprises up-regulating BC200 RNA in a patient. Such up-regulation may comprise administering to a patient in need of such treatment, a therapeutically effective amount of BC200 RNA. Alternatively, a gene therapy construct having a DNA or RNA corresponding to BC200 operably linked to a promoter which functions in a subject to drive expression of BC200 RNA may be administered to a patient. Modifications to the nucleotide sequence of BC200 RNA (SEQ ID NO:1) which modifications still allow BC200 RNA to maintain the characteristic property of repressing translation initiation are within the scope of the present invention. Such modifications include insertions, deletions and substitutions of one or more nucleotides.

The present invention further provides kits for use in practicing the present invention. In one embodiment, a kit comprises at least one subject antisense oligonucleotide and a buffer solution or a pharmaceutically acceptable carrier. The buffer or pharmaceutically acceptable carrier may be packaged either separately from, or admixed with, the subject antisense molecule(s). For example, a kit may comprise a first container comprising a subject antisense molecule e.g., as a lyophilized powder. A second container may contain a pharmaceutically acceptable carrier for use in mixing with the antisense molecule in order to make a formulation in an acceptable dosage for administering to a subject The kit preferably also contains instructions on formulation in order to arrive at a dosage range hereinbefore described. The kit may also contain other materials useful for practicing the present invention such as, e.g., syringes, needles, etc.

The invention is further illustrated by the following specific examples which are not intended in any way to limit the scope of the invention.

### EXAMPLES

In accordance with the present invention, BC1 RNA, the rodent analog to primate BC200 RNA, has been identified as a specific repressor of translation in dendrites. (It should be noted that sequence similarity between rodent BC1 RNA and primate BC200 RNA (Tiedge et al., 1993) is restricted to the 3′ domain and the central A-rich domain.) BC1 RNA is a non-translatable small neuronal RNA that does not contain a protein coding sequence (reviewed by Brosius and Tiedge, 1995; Brosius and Tiedge, 2001). It has previously been localized to dendrites (reviewed by Brosius and Tiedge, 2001) where it was found enriched in postsynaptic compartments, colocalized with a subset of neuronal mRNAs that are selectively delivered to dendrites (Chicurel et al., 1993). It has previously been shown that this RNA is specifically and rapidly transported to dendrites (Muslimov et al., 1997), and that somatodendritic BC1 expression levels are subject to activity-dependent modulation (Muslimov et al., 1998). It was on the basis of such and other evidence that BC1 RNA was hypothesized to function as a translational modulator (Brosius and Tiedge, 2001).

As set forth below in greater detail, BC1 RNA is a specific repressor of translation initiation both in cap-dependent and internal entry modes. The combined data indicate that non-translatable BC1 RNA plays a functional role in translational control of gene expression in neurons.

### Example 1

### Materials and Methods

RNAs. Plasmid pBCX607 was used to generate full length BC1 RNA as described before (Cheng et al., 1996; Muslimov et al., 1997). Plasmids pSP6-U4 and pSP6-U6 (Hausner et al., 1990) were used for the in vitro transcription of U4 and U6 snRNAs, respectively, as described (Muslimov et al., 1997). Yeast tRNA was purchased from Sigma (St. Louis, Mo.). Plasmid pTub-A98/TA2 was kindly provided by Dr. J. Brosius. In this vector, the full-length α-tubulin cDNA insert is immediately followed by an uninterrupted stretch of 98 A residues. It was linearized with XbaI or XhoI, and in vitro transcribed with T7 RNA polymerase, to yield programming mRNA encoding α-tubulin either with or without a 3′ 98-residue poly(A) tail, respectively.

Plasmid pBDCG (kindly provided by Dr. J. Carson) was used to produce polyadenylated BFP/EMCV-IRES/GFP (Blue Fluorescent Protein/Encephalomyocarditis Virus-Internal Ribosome Entry Site/Green Fluorescent Protein) dicistronic mRNA as described (Kwon et al., 1999). To generate a monocistronic version, plasmid pMCG was derived from pBDCG by partial digestion with XbaI and XmaI to remove segment nt 28-753. It was linearized with SapI and transcribed with SP6 RNA polymerase to produce polyadenylated EMCV-IRES/GFP mRNA. Plasmid pCSFV(1-442).NS' (A) was used to generate polyadenylated CSFV-IRES/NS' (Classical Swine Fever Virus-IRES/truncated influenza virus non-structural protein) programming mRNA. Derived from plasmid pCSFV(1-442).NS' (Pestova et al., 1998) by insertion of an A98-segment at position 1305, it was linearized with EcoRI for in vitro transcription with T7 RNA polymerase. All programming mRNAs were used polyadenylated, unless noted otherwise. Whenever desired, mRNAs were capped by in vitro transcription in the presence of 0.3 mM m⁷G(5′)ppp(5′)G (Stratagene, La Jolla, Calif.).

Expression and Purification of recombinant proteins. Recombinant eIF4A was expressed from plasmid pET(His₆-eIF4A) in Escherichia coil BL21(DE3) and purified as described (Pestova et al., 1996a). Recombinant eIF4G (central domain, aa 697-1076) was analogously generated from pET28(His₆-eIF4G₆₉₇₋₁₀₇₆) (Lomakin et al., 2000).

Recombinant poly(A)-binding protein (PABP) was generated from vector pET3B.PABP-His as described before (Khaleghpour et al., 2001). A C-terminal domain (aa 462-633) of poly(A)-binding protein (PABP) was generated from vector pGex2T.PABPaa462-633 (Imataka et al., 1998). Analogously, an N-terminal domain (aa 1-182) of PABP, containing RNA recognition motif (RRM) domains I and 2, was generated from vector pGex2T.PABPaal-182. Expressed as glutathione S-transferase (GST) fusion proteins, PABP domains were purified on glutathione-Sepharose beads (Amersham Biosciences, Piscataway, N.J.) as described (Smith and Johnson, 1988).

Translation assays. Rabbit reticulocyte lysates were purchased from Ambion (Austin, Tex.) or Roche (Indianapolis. Ind.), and in vitro translation reactions were performed according to the instructions of the manufacturer. Lysate, reaction buffer, ³⁵S-methionine (˜1200 Ci/mmol, from NEN, Boston, Mass.), and respective programming IRNA were incubated for 1 hour at 30° C. in the presence of BC 1 RNA or other small RNAs, as indicated. Reaction mixtures were treated with 0.1 mg/ml RNaseA for 10 min, and translation products were separated by SDS-PAGE, using 10% acrylamide gels. Gels were dried and subjected to autoradiography to visualize protein bands. Signal intensities of bands were quantified using a Storm 860 phosphorimaging system with ImageQuant software (Molecular Dynamics, Sunnyvale, Calif.).

The integrity of programming mRNAs that were used in this work was verified in time-course experiments with ³²P-labeled transcripts under otherwise identical reaction conditions. No RNA degradation was observed in any of these control experiments.

Analysis of ribosomal complexes. To analyze 48S and 80S complexes, sucrose density gradient centrifugation was used according to previously established protocols (Gray and Hentze, 1994; Pestova et al., 1996a). In vitro translation reactions were performed as described above, except that the reaction mixtre did initially not contain mRNA and that methionine was not radiolabeled. The reaction mixture was pre-incubated at 30° C. for 15 min with translational inhibitor guanylyl imidodiphosphate (GMP-PNP; 1.2 mM) or cycloheximide (0.8 mM). Small RNAs (e.g. BC1 RNA, U4 RNA) were used at 600 nM. Subsequently, ³²P-labeled programming mRNA (50 ng) was added, and incubation continued for another 5 min at 30° C. Complexes were resolved by centrifugation through a 5% to 25% sucrose gradient in SG buffer (100 mM KCl, 2 mM DTT, 2 mM magnesium acetate, 20 mM Tris-HCl, pH 7.5) for 3 hours at 4° C. at 30,000 rpm with a Beckman SW41 rotor. 25 fractions were collected per tube, starting from the bottom. The radioactivity of fractions was determined by Cerenkov counting.

Electrophoresis mobility shift assay (EMSA). ³²P-labeled RNA probes (50,000 cpm per reaction, ˜10 ng) were heated for 10 min at 70° C., cooled for 5 min at room temperature, and then incubated together with proteins in binding buffer (300 mM KCl, 5 mM MgCl₂, 2 mM DTT, 5% glycerol, 20 mM HEPES, pH 7.6) for 20 min at room temperature. If unlabeled competitor RNAs were used, they were treated analogously but pre-incubated with proteins for 10 min before labeled RNAs were added to the reaction. Reaction time was increased to 40 min if simultaneous binding to more than one protein was analyzed. RNA-protein complexes were subsequently resolved on 5% polyacrylarnide gels (60:1 polyacrylamide:bis-acrylamide) and analyzed by autoradiography as described (Gu and Hecht, 1996; Thomson et al., 1999).

Brain extracts. Brains were dissected from adult Sprague-Dawley rats and were immediately frozen in liquid nitrogen. Brains were resuspended in 2 ml/brain of buffer A [100 mM NaCl, 0.5 mM dithiothreitol, 3 mM MgCl₂, 0.5 mM phenylmethylsulfonyl fluoride (PMSF), 0.5 μg/ml leupeptin, 1 μg/ml aprotinin, 50 mM Tris-HCl, pH 8.0), and homogenized slowly on ice with a motor-driven homogenizer (Kontes, Vineland, N.J.). The homogenate was centrifuged at 5,000 g for 15 min. The supematant was mixed with 0.1 volume of buffer B (2.5 M NaCl, 500 mM Tris-HCl, pH 8.0). After further centrifugation at 14,000 g for one hour at 4° C., the supematant was snap-frozen in liquid nitrogen and stored at −70° C.

Immunodepletion of brain extracts. Brain extracts (60 μl) were incubated with 20 μl of anti-GST-PABP (aa 462-633; Imataka et al., 1998) for 3 hours at 4° C. with gentle rotation. Subsequently, 15 μl of protein-A agarose (Roche, Indianapolis, Ind.) suspension was added to the mixture and was incubated, with rotation, at 4° C. overnight. Complexes were collected by centrifugation at 12,000 g for 20 seconds (Zhang et al., 2001). The immunodepleted brain extracts were then used for electrophoretic mobility shift assays (EMSAs) as described above.

Supershift assay. ³²P-labeled in vitro transcribed BC1 RNA (50,000 cpm per reaction, ˜1 ng) was heated for 10 min at 70° C. and cooled for S min at room temperature. The RNA was then incubated with brain extract (30-40 μg) or immunodepleted brain extract in binding buffer for 20 min at room temperature. In competition experiments, unlabeled BC1 RNA (2000-fold excess) was added 10 min before the binding reaction. Mixtures containing brain extract were then incubated with an anti-GST-PABP antibody (raised against a fusion protein containing PABP aa 462-633; Imataka et al., 1998) or an anti-GST control antibody for 3 hours at room temperature. To minimize unspecific binding, samples were incubated with heparin (5 mg/ml) for 10 min at room temperature. As in EMSA, complexes were resolved on 4% native polyacrylamide gels and analyzed by autoradiography.

Immunocvtochemistry with hippocampal neurons in primary culture. Immunocytochemistry was performed as described (Tiedge and Brosius, 1996). Primary antibodies were used at tle following dilutions: anti-eIF4A, 1:50; anti-eIF4G, 1:50; anti-PABP, 1:50; anti-synaptophysin, 1:500. Polyclonal anti-eIF4A, anti-PABP and anti-eIF4G antibodies have been described before, and their respective specificities established (Wakiyama et al., 2000). A monoclonal anti-synaptophysin antibody was purchased from Synaptic Systems, Göttingen, Germany. Secondary antibodies were used as follows: biotinylated anti-rabbit (Amersham), 1:200; anti-mouse labeled with fluorescein isothiocyanate (Jackson ImmunoResearch, West Grove, Pa.), 1:25. Biotinylated secondary antibodies were decorated with streptavidin-conjugated rhodamine (5 μg/ml, Jackson). Control experiments to ascertain unspecific background labeling were performed as follows: (1) In the case of polyclonal antibodies, pre-immune or non-immune serum was substituted for the primary antibody; (2) In the case of antibodies directed against GST fusion proteins, an anti-GST antibody was used as a primary antibody; (3) Background labeling was further ascertained by incubation in the absence of a primary antibody. Confocal images were acquired with a Radiance 2000 Plus confocal laser scanning microscope (Bio-Rad, San Francisco, Calif.) attached to an Axioskop 2 microscope (Zeiss, Thornwood, N.Y.).

### Example 2

### Results

BC1 and BC200 RNA are Specific Repressors of Translation

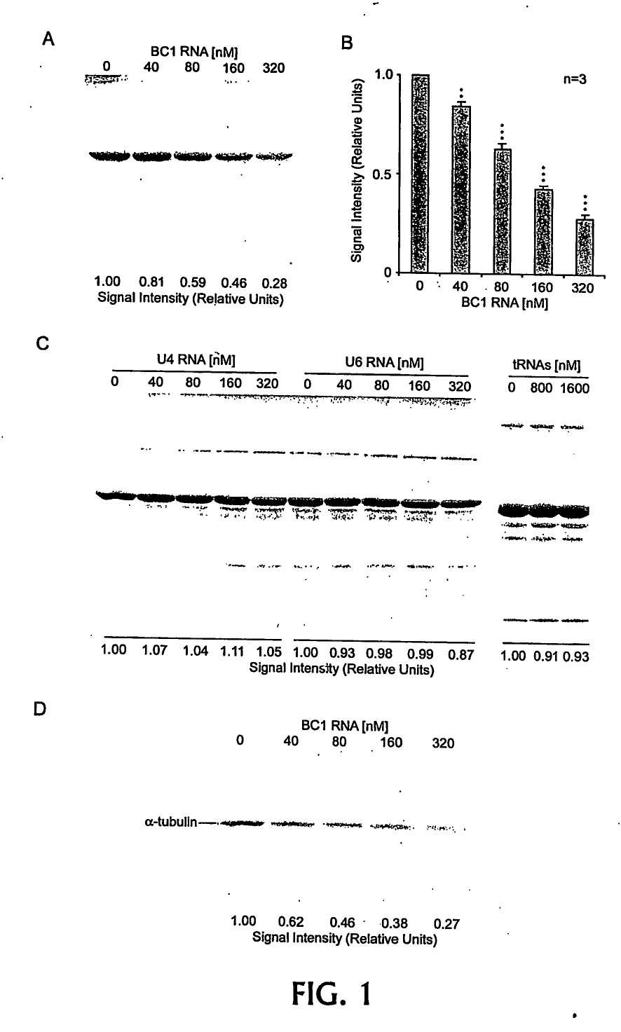

The rabbit reticulocyte lysate (RRL) cell-free system was used to probe the competence of BC1 RNA as a modulator of translation. In untreated RRLs (i.e. reticulocyte mRNA transcripts not removed by nuclease), translation of endogenous mRNAs was inhibited by BC1 RNA in a concentration-dependent manner (FIG. 1A,B). Results from these experiments were quantified by phosphorimaging. Analysis of several experiments showed that the presence of BC1 RNA at a concentration of 320 nM resulted in a decrease of translation efficiency by 70-80%. Such a reduction was observed with all protein bands that were resolved by SDS PAGE, a result indicating that BC1-mediated translational repression was not restricted to particular mRNAs. However, in clear contrast to BC1 RNA, other small non-translatable RNAs (e.g. U4 and U6 snRNAs, tRNAs), used at similar or higher concentrations, had no effect on translation efficiency (FIG. 1C). The results demonstrate that BC1 RNA is a specific repressor of translation that is effective in the sub-micromolar concentration range.

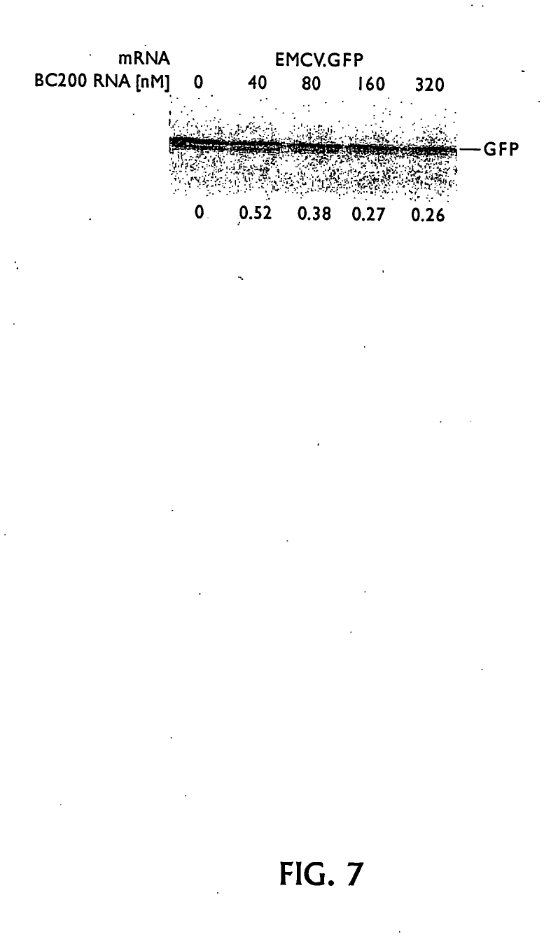

These results were confirmed with lysates in which endogenous RRL transcripts had been removed by nuclease treatment prior to translation experiments. Using capped and polyadenylated α-tubulin mRNA as a programming mRNA in these experiments, we established that BC1 RNA (but not nuclear U4 RNA or other control RNAs) inhibited cap-dependent translation to the same degree and in the same sub-micromolar concentration range as shown above (FIG. 1D). Uncapped or non-adenylated programming mRNAs were not efficiently translated; translation of capped but non-adenylated α-tubulin mRNA appeared to be less susceptible to BC1-mediated inhibition than capped and polyadenylated progranining mRNA although this could not be reliably established due to lower overall translational efficiencies. All subsequent experiments were therefore performed with polyadenylated progranmming mRNA, unless noted otherwise. Furthermore, BC200 RNA, the primate counterpart of rodent BC1 RNA (Tiedge et al., 1993), used in the same nanomolar concentration range, was found to inhibit translation as effectively as BC1 RNA (see FIG. 7).

In summary, the above data indicate that BC1 RNA and BC200 RNA act as specific repressors of translation.

BC1 RNA Inhibits Formation of the 48S Preinitiation Complex

Eukaryotic translation can be subdivided into the three sequential phases of initiation, elongation, and termination. Frequently, it is the initiation phase that is targeted in translation regulation mechanisms (Gingras et al., 1999).

It was hypothesized that in repressing translation, BC1 RNA interacts with the translational machinery at the level of initiation. This hypothesis was tested as follows.

Cap-dependent translation initiation typically begins with the assembly of the 40S small ribosomal subunit, eukaryotic initiation factor (eIF) 1A, eIF3, and an eIF2/GTP/Met-tRNAᵢ complex, to form a 43S preinitiation complex. In the next step, the 43S complex is recruited to the mRNA and translocates (‘scans’) to the AUG start codon where it forms a stable 48S preinitiation complex. This recruitment step, often the rate-limiting one in initiation and frequently also the target of regulation, is mediated by the eIF4 group of factors. The m⁷GpppN cap at the 5′ end of the mRNA is recognized by the eIF4E subunit of eIF4F. eIF4E is bound to eIF4G, a central coordinator of initiation that also associates with eIF3 and eIF4A, an RNA helicase that unwinds secondary structure. (The heterotrimeric complex of eIF4A, eIF4E, and eIF4G constitutes eIF4F.) Finally, after release of initiation factors from the 48S preinitiation complex, the 60S ribosomal subunit joins to form the 80S complex (for reviews, see Gingras et al., 1999; Hershey and Merrick, 2000; Pestova et al., 2001; Dever, 2002).

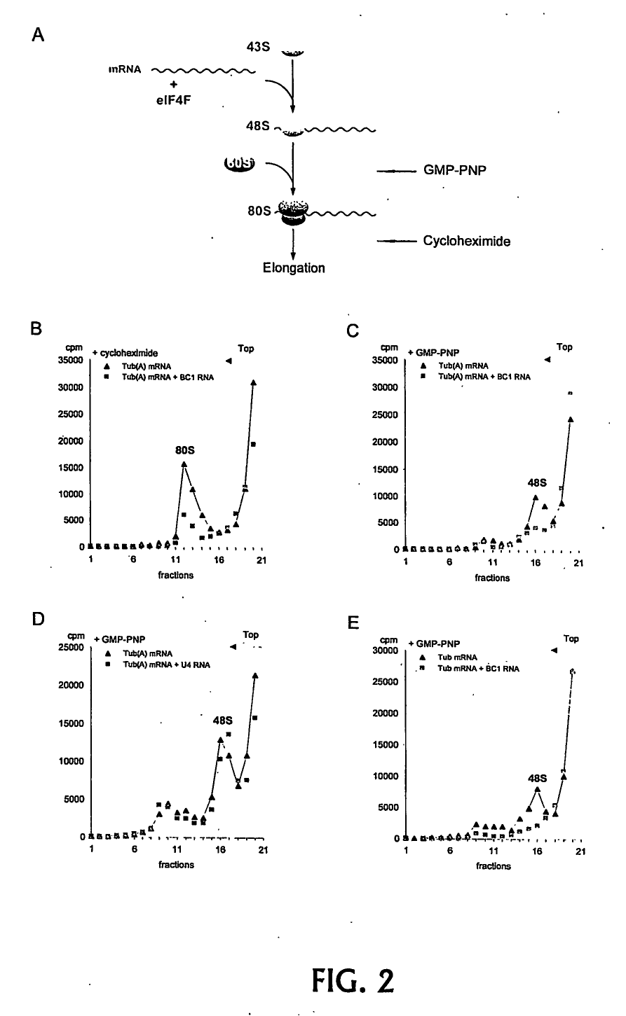

To dissect functional interactions of BC1 RNA with the translation initiation mechanism, different stages in translation initiation were visualized by arresting the mechanism at that stage, and by subsequently resolving stable complexes by sucrose density gradient centrifugation As described previously (Gray and Hentze, 1994), recruited 43S preinitiation complexes will stall at the initiator AUG, and 48S complexes will therefore accumulate, if the subsequent step of initiation factor dissociation (which depends on the hydrolysis of GTP bound to eIF2) is blocked by the non-hydrolyzable GTP analog guanylyl imidodiphosphate (GMP-PNP). Analogously, 80S ribosomal initiation complexes can be detected by using cycloheximide to inhibit elongation: ribosomes will be arrested at the start site, resulting in the accumulation of 80S complexes (see FIG. 2A for a schematic illustration).

Cycloheximide was used to visualize assembly of 80S complexes with a capped programming mRNA encoding α-tubulin (FIG. 2B). Full-length BC1 RNA, used at 600 nM, significantly reduced 80S complex formation, indicating that translation initiation was inhibited at or before this step. GMP-PNP was then used to visualize formation of 48S preinitiation complexes. As with 80S complex formation, the presence of 600 nM BC1 RNA resulted in a significant reduction of 48S complex assembly (by 81% on average; FIG. 2C). In contrast to BC1 RNA, U4 RNA at the same concentration had no effect on the formation of 48S complexes (FIG. 2D). These data confirm that the BC1-mediated inhibition of initiation complex formation was specific. Finally, no difference was observed in the extent of BC1-mediated inhibition of 48S complex formation depending on whether the programming α-tubulin mRNA was polyadenylated or non-adenylated (FIG. 2E). These results suggest the inhibition of translation initiation by BC1 RNA is not dependent on the adenylation status of the programming mRNA.

Taken together, these results indicate that BC1 RNA specifically represses formation of the 48S preinitiation complex (and, consequently, of the 80S complex) and are consistent with the notion that BC1 RNA inhibits recruitment of the 438 complex to the mRNA, and/or its translocation to the AUG start site.

BC1 RNA Represses Translation Through Interaction with Initiation Factors of the eIF4 Group

Having shown that BC1 RNA inhibits assembly of the 48S preinitiation complex, the target site(s) of BC1 RNA in that part of the translation initiation pathway that leads to 48S complex formation was identified. A functional test was utilized which took advantage of different types of viral internal ribosome entry site (IRES) translation initiation mechanisms.

Internal ribosome entry provides an alternative to the cap-dependent initiation mechanism: the small ribosomal subunit binds to an IRES, either at or upstream of the AUG start codon, in an end-independent fashion (reviewed by Jackson, 2000; Hellen and Sarnow, 2001; Pestova et al., 2001). Viral internal ribosome entry initiation mechanisms differ from each other in their need for canonical initiation factors. Two major subtypes of viral internal entry mechanisms can be distinguished. The first one is exemplified by the encephalomyocarditis virus (EMCV) and other picomavirus IRESs. Formation of the 48S complex at the EMCV IRES requires the same set of canonical initiation factors as the cap-dependent mechanism except for eIF4E, the cap-binding protein (Pestova et al., 1996a; Pestova et al., 1996b). Translation commences at the AUG at the 3′ border of the IRES: thus, no scanning is necessary, but eIF4A is required to melt mRNA secondary structure for effective ribosomal recruitment. A second subtype of internal entry, exemplified by the hepatitis C virus (HCV) IRES and the classical swine fever virus (CSFV) and related pestivirus IRESs, employs a much simpler mechanism (Pestova et al., 1998). This type of IRES binds directly to the 40S ribosomal subunit in a mechanism that does not require any of the factors of the eIF4 group.

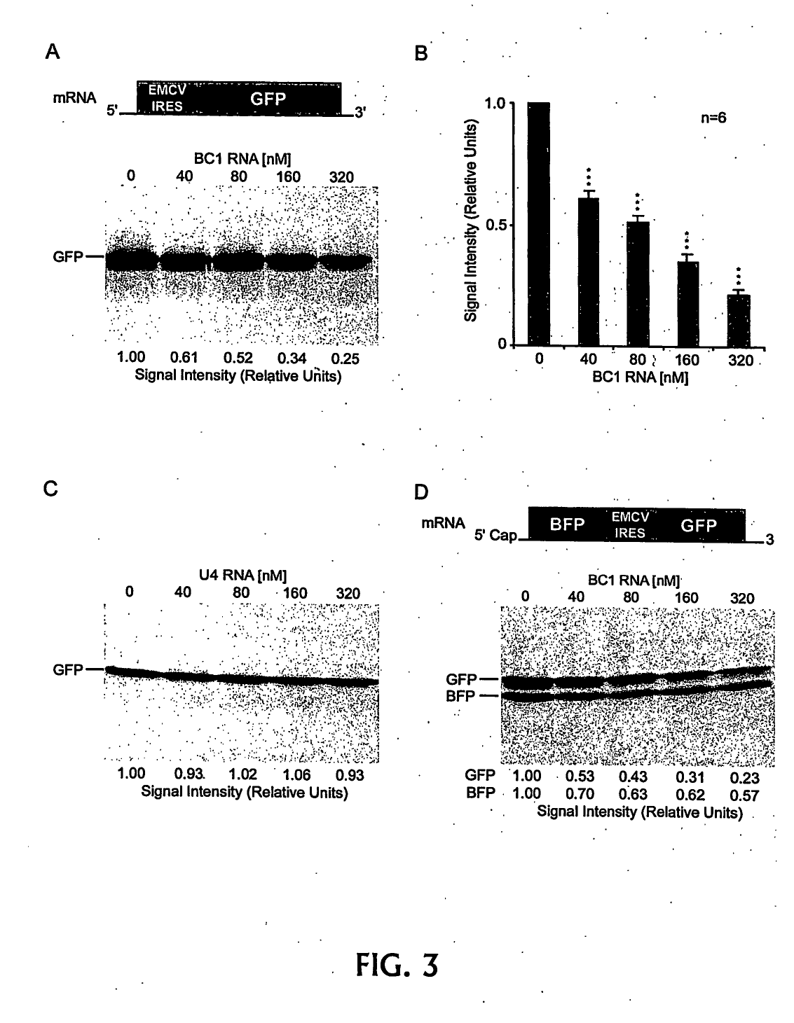

The two described internal entry mechanisms were used for a functional dissection of translation initiation repression by BC1 RNA First, experiments were conducted to determine if such repression was cap-dependent An uncapped programming mRNA (encoding Green Fluorescent Protein, GFP) was used in which internal entry was mediated by the EMCV IRES. BC1 RNA effectively repressed translation of this mRNA (FIG. 3A). Phosphorimaging quantification of 6 experiments showed that on average, BC1 RNA decreased translation efficiency by about 79% at 320 nM (FIG. 3B). This reduction is very similar in extent to the one observed above for capped programming mRNAs. As in cap-dependent translation, U4 RNA had no effect on translation efficiency (FIG. 3C). Similar results were obtained with other programming mRNAs and with dicistronic constructs. In the example shown in FIG. 3D, the first cistron was preceded by a 5′ cap whereas the second cistron was preceded by an EMCV IRES. BC1 RNA inhibited both cap- and IRES-mediated translation in this system. Translation from the IRES-dependent cistron, being more efficient in the absence of BC1 RNA, was also more susceptible to BC1-mediated repression. Accordingly, the EMCV IRES has a higher dependence on a factor/activity that is inhibited by BC1 RNA. It is interesting to note in this context that translation mediated by this IRES is also more strongly inhibited by trans-dominant eIF4A mutants than cap-dependent translation (Pause et al., 1994). Finally, analogous experiments with human BC200 RNA revealed that this RNA repressed translation in very much the same fashion. Translation initiated by internal entry at the EMCV IRES was inhibited by BC200 RNA by 73% at 270 nM (see FIG. 7).

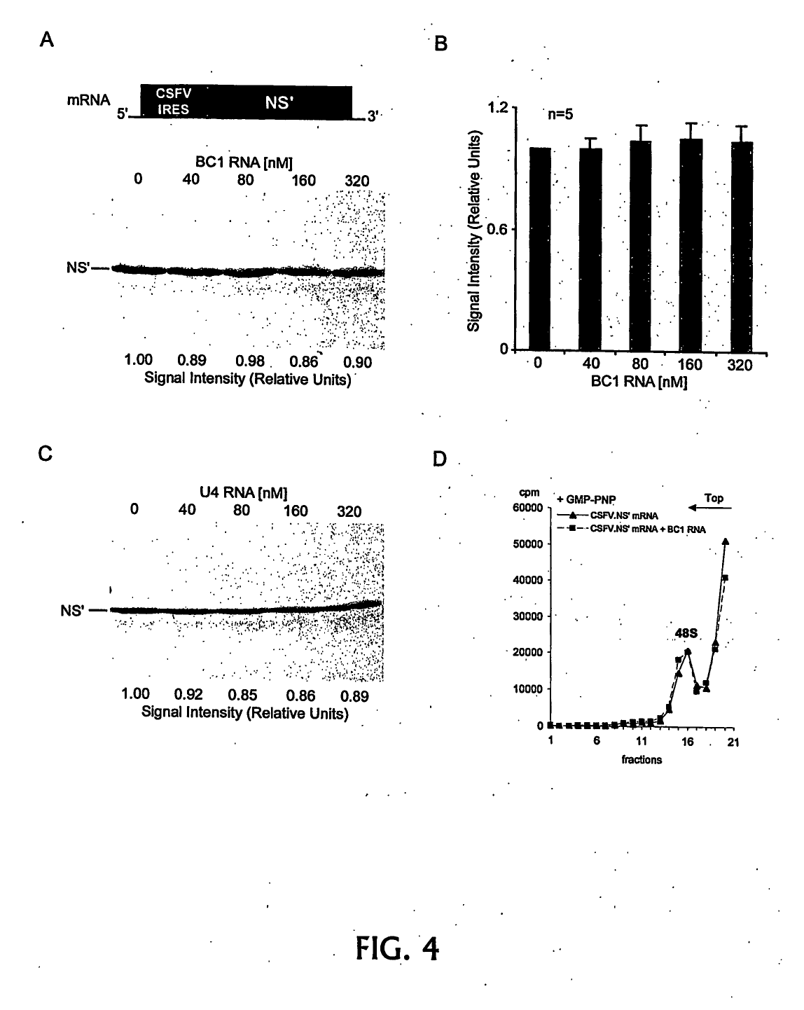

BC1-mediated translational repression, the results indicate, is not cap/eIF4E-dependent as translation initiated through internal entry via the EMCV IRES mechanism is equally inhibited. Additional experiments were conducted to determine whether or not other members of the eIF4 family of translation initiation factors were required for BC1-mediated translational repression utilizing the CSFV IRES system. FIG. 4A shows that BC1 RNA was not effective in repressing translation if internal entry was mediated by the CSFV IRES. Quantification by phosphorimaging revealed no significant change in translational efficiency with increasing concentrations of BC1 RNA (FIG. 4B). Control RNAs such as U4 RNA (FIG. 4C) were equally ineffectual. Accordingly, translation initiation by internal entry using the CSFV IRES mechanism effectively bypasses BC1-mediated translational repression.

These results were confirmed by sucrose density gradient centrifugation analysis. BC1 RNA was found not to repress formation of either 48S complexes (FIG. 4D) or 80S complexes if internal entry occurred at the CSFV IRES. This result confirms that translation initiated via the CSFV IRES mode is refractory to BC1-mediated repression Mechanisms that are common to both the CSFV IRES and the EMCV IRES mode can therefore be ruled out as candidate targets for BC1-mediated translational repression. These include all elongation and termination steps as well as most steps in the initiation pathway—such as, for example, formation of the ternary eIF2/GTP/Met-tRNAᵢ complex, prerequisite for 48S complex assembly (reviewed by Hellen and Samow, 2001; Pestova et al., 2001).

Initiation on the CSFV IRES differs from both EMCV IRES mediated and cap-dependent initiation in that there is no requirement for any of the members of the eIF4 group of factors (Pestova et al., 1998). Of these factors, eIF4G and eIF4A are required for 48S complex assembly in the EMCV-type internal entry mode, but not in the CSFV-type internal entry mode (Pestova et al., 1996a; Pestova et al., 1998). In addition, poly(A)-binding protein (PABP) also qualifies as a potential BC1 target as it enhances initiation mediated by the EMCV IRES (Michel et al., 2001; Svitlcin et al., 2001).

Formation of the 48S preinitiation complex is the rate-limiting step in translation initiation under most circumstances (reviewed by Gingras et al., 1999; Hershey and Merrick, 2000). The data indicate that BC1-mediated translational repression operates through the eIF4 family of initiation factors because internal initiation by the CSFV IRES mechanism, which does not require any of these factors, effectively bypasses this repression. A key factor in the recruitment of the 43S preinitiation complex to the mRNA is eIF4F, a heterotrimeric complex composed of eIF4E, a cap-binding protein, eIF4A, an ATP-dependent RNA helicase, and eIF4G, a large scaffolding protein (reviewed by Gingras et al., 1999; Jackson, 2000; Pestova et al., 2001). The data reported here show that BC1-mediated repression is cap- (and therefore eIF4E-) independent.

eIF4A and PABP Interact Directly with BC1 RNA

Functional analysis was used to narrow potential target sites for BC1-mediated inhibition in the translation initiation pathway and, consequently, potential BC1 interacting factors in the translation initiation machinery. Biochemical methods were utilized for a direct analysis of BC1-protein interactions with those candidates.

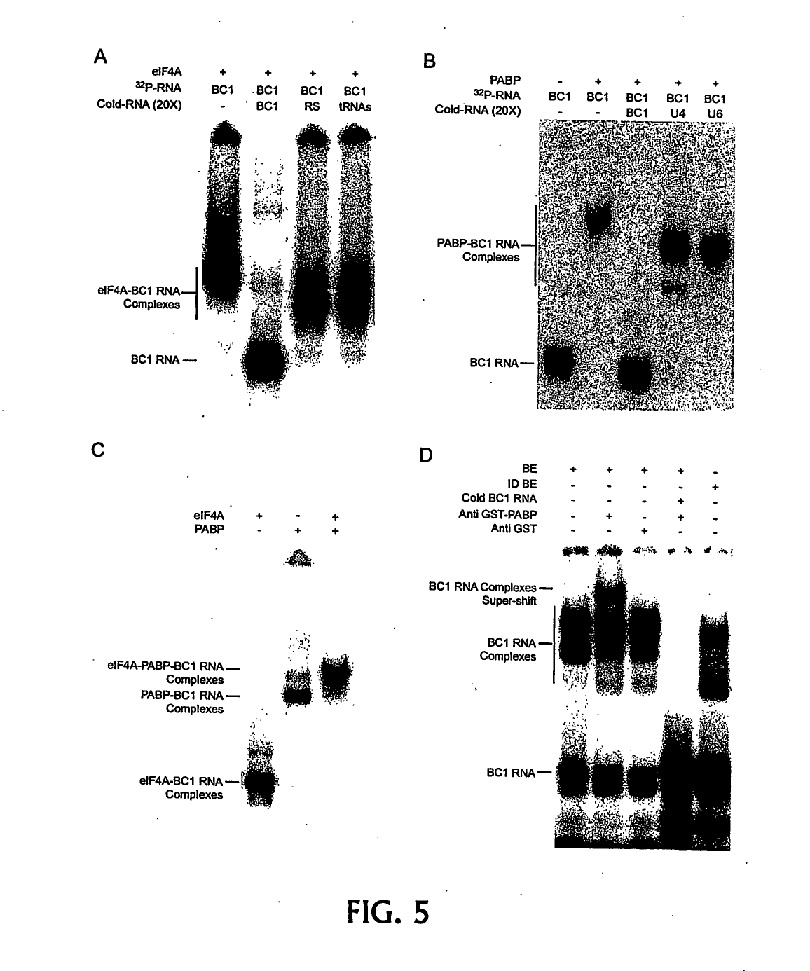

Using electrophoretic mobility shift assays (EMSAs) with recombinant proteins, binding of BC1 RNA to eIF4A, eIF4G, and PABP was probed. Since the central domain of eIF4G has previously been shown to bind to the EMCV IRES (Pestova et al., 1996b), potential interactions of BC1 RNA with this domain were examined. No specific binding of BC1 RNA to the central eIF4G domain was detected (aa 697-1076). In contrast, EMSA analysis revealed specific binding of BC1 RNA to eIF4A (FIG. 5A). Specificity was demonstrated by the fact that pre-incubation with unlabeled BC1 RNA effectively abolished the mobility shift. Conversely, unlabeled irrelevant RNAs such as random-sequence vector RNA or tRNAs were not effective in competing with BC1 RNA for binding to eIF4A in these assays (FIG. 5A). In the presence of such non-competing RNAs, the eIF4A-induced mobility shift was resolved as a duplex band. This observation indicates that under these conditions, two BC1/eIF4A complexes were migrating at slightly different mobilities.

In addition, BC1 RNA was found to specifically bind to PABP (FIG. 5B). Again, specificity was ascertained in EMSA competition experiments in which unlabeled BC1 RNA effectively competed for binding whereas irrelevant RNAs did not Simultaneous exposure of BC1 RNA to both eIF4A and PABP in EMSA experiments produced a larger shift than exposure to either eIF4A or PABP alone (FIG. 5C), indicating that binding of these two proteins to BC1 RNA was not mutually exclusive. In addition, using an antibody specific for PABP, the mobility shift that is observed with BC1 RNA in rat brain extracts was specifically ‘supershifted’ to further reduced mobility (FIG. 5D). Conversely, if the same antibody was used to immunodeplete brain extracts of PABP, the mobility shift of BC1 RNA was now predominantly observed at increased mobility (FIG. 5D). Taken together, the results indicate that BC1 RNA interacts specifically with eIF4A and PABP.

eIF4A, eIF4G, and PABP are Localized in Dendrites

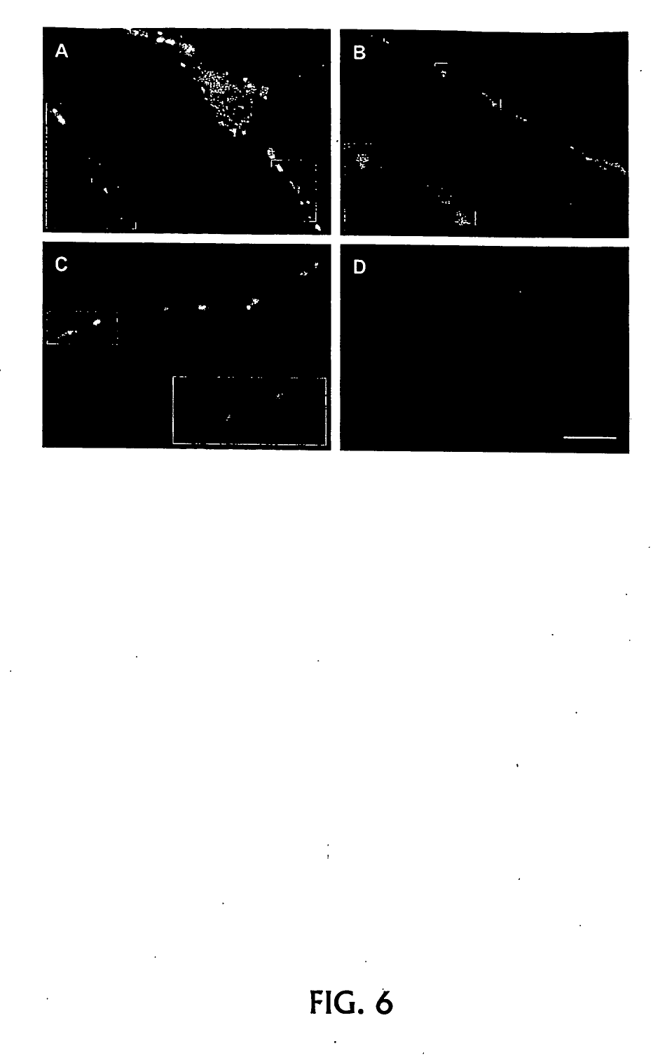

Since BC1 RNA is targeted to dendrites, any interaction with eIF4A and PABP would obviously require the presence in dendrites of these proteins as well. In addition, eIF4G would also be needed in its role of a scaffolding protein that interacts with both eIF4A and PABP (reviewed by Gingras et al., 1999; Jackson, 2000; Dever, 2002). The presence of these three proteins in dendrites was probed using immunocytochemistry in conjunction with confocal laser scanning microscopy (CLSM) to hippocampal neurons in culture (Tiedge and Brosius, 1996). The results presented in FIG. 6 illustrate that eIF4A, eIF4G, and PABP were detectable in dendrites at substantial levels. (No significant labeling was detected along axonal shafts for any of these factors.) Throughout dendrites, labeling patterns for all three proteins were of heterogeneous, particulate nature, often giving a punctate appearance. On average, such labeling clusters were less frequently observed in distal dendritic segments than in proximal segments. The results indicate that eIF4A, eIF4G, and PABP are distributed along dendrites in a heterogeneous, clustered fashion.

Immunocytochemical experiments were performed in dual-labeling mode, using in parallel an antibody against synaptophysin, a marker protein for synaptic vesicles and thus for presynaptic specializations (Jahn et al., 1985), to determine whether or not these dendritic clusters were associated with synaptic structures. This antibody has previously been shown to identify presynaptic specializations as discrete puncta in mature hippocampal neurons in culture (Fletcher et al., 1991; Fletcher et al., 1994). Using CLSM, such puncta were found to be prominently displayed along dendritic extents, typically at decreasing frequency in more distal segments (FIG. 6). Subpopulations of eIF4A, eIF4G, and PABP labeling clusters were seen in spatial association with synaptophysin puncta. Such association was best observed in distal dendrites where cluster densities were not so high as to obscure resolution by excessive overlap (FIG. 6). Red (eIF4A, eIF4G, or PABP) and green (synaptophysin) labeling clusters were often seen in direct apposition to each other, the latter typically of more superficial appearance. Some, but not all, apposing red/green puncta pairs apparently overlapped to some degree, evidenced by narrow yellow interface areas. Since green puncta identify axonal presynaptic specializations, it is concluded that such apposing red clusters correlate with postsynaptic dendritic compartments.

In summary, the results indicate a differential intradendritic localization of eIF4A, eIF4G, and PABP clusters, with some of those clusters positioned in postsynaptic microdomains underneath, or in direct vicinity of, presynaptic axonal specializations. Such synapse-associated clusters in dendrites can serve in the local synthesis of dendritic proteins (such as CaMKIIα; Burgin et al., 1990) that are enriched in postsynaptic compartments whereas extrasynaptic eIF4A, eIF4G, and PABP clusters preferentially participate in the synthesis of dendritic proteins (such as MAP2; Garner et al., 1988) that are not synapse-associated.

Experimental use of internal ribosome entry mechanisms and sucrose density gradient centrifugation showed that BC1-mediated repression targets translation at the level of initiation. Specifically, BC1 RNA inhibited formation of the 48S preinitiation complex, i.e. recruitment of the small ribosomal subunit to the mRNA. However, 48S complex formation that is independent of the eIF4 family of initiation factors was found to be refractory to inhibition by BC1 RNA, a result that implicates at least one of these factors in the BC1 repression pathway. Biochemical experiments indicated a specific interaction of BC1 RNA with eIF4A, an RNA unwinding factor, and with poly(A)-binding protein (PABP). Both proteins were found enriched in synaptodendritic microdomains. Significantly, BC1-mediated repression was shown to be effective not only in cap-dependent translation initiation but also in eIF4-dependent internal initiation.

The results indicate BC1 RNA is a mediator of translational control in local protein synthesis in nerve cells. Accordingly, its human analog, BC200 RNA, is a suitable target for antisense treatment of neurological disorders characterized by an increase in BC200 RNA levels.

With the significance of functional, non-translatable RNAs in cellular structure and function being increasingly appreciated, the traditional view of RNAs as mere passive carriers of information is in obvious need of amendment. Non-translatable RNAs have been implicated in various cellular functions (reviewed by Storz, 2002); microRNAs, for example, may participate in translational control, albeit in mechanisms that are clearly distinct from the BC1 pathway. Functional RNAs may exist in much larger numbers than hitherto assumed, and it is likely that genes encoding such RNAs, far from being mere remnants of an early RNA world, are continually being generated in eukaryotic species (Brosius and Tiedge, 1996; Kuryshev et al., 2001; Eddy, 2002; Wang et al., 2002). Therefore, non-translatable RNAs in nerve cells not only function as determinants of neuronal fluctionality and plasticity, but at the same time serve as a driving force in neural species diversification.

### Example 3

### Materials and Methods

Surgery and Electrophysiology. Standard in vivo LTP and kindling protocols were used (Cain et al., 1992; Steward et al., 1998). Male Sprague-Dawley rats (22 total, 350-600 g) were anesthetized with urethane (1 g/kg administered i.p.). After an appropriate anesthetic level was attained, the animals were placed in a stereotaxic frame, the scalp incised, retracted, and lambda and bregma were placed on the same horizontal plane. Animals were implanted with monopolar stimulating and recording electrodes composed of single Teflon coated stainless steel wires, cut flush at the tips (diameter 65 γm). Both stimulating and recording electrodes were referenced to stainless steel screws implanted in the skull.

Animals for the LTP experiments were implanted unilaterally on the left side with stimulating and recording electrodes in the perforant path and dentate gyrus, respectively. Perforant path stimulating coordinates were −0.5 mm posterior and 4.5 mm lateral relative to the lambda suture intersection, while dentate gyrus recording coordinates were −3.8 mm posterior and 2.5 mm lateral relative to the bregma suture intersection. Animals for the seizure experiments were implanted unilaterally on the right side with stimulating and recording electrodes in the stratum radiatum of CA3 and CA1, respectively. CA3 stimulating coordinates were −3.5 mm posterior and lateral to the bregma suture intersection, while CA1 recording coordinates were −3.8 mm posterior and 2.5 mm lateral to the bregma suture intersection.

Final depth positioning of all electrodes was done under physiological control, and set to optimize the response from the appropriate implanted pathways. Evoked potential recordings were amplified by 10000, band-pass filtered from 1 Hz to 10 KHz (A-M Systems Model No. 1700 differential AC amplifier, Carlsborg, Wash.), digitized at 20 KHz and stored to disk on a PC. Electroencephalogram (EEG) was similarly amplified but band-pass filtered from 1 Hz to 200 Hz and digitized at 400 Hz. Evoked potential responses were analyzed offline for field excitatory postsynaptic potential (fEPSP) slope and population spike amplitude. The fEPSP slope was measured as the rise over the run of a 1 rnsec-segment just before the emergence of the population spike (initial slope). The population spike amplitude was measured as the distance in mV from the initial deflection to the maximal deflection of the population spike. Secondary ADs were not observed.

Evoked potential test pulses were biphasic (0.1 msec/phase, negative phase leading). Once recordings were stable, input/output (I/O) curves were obtained and used to determine both baseline and tetanization intensities of stimulation current. During implantation and I/O curve determination, test pulse frequencies were at approximately 0.1 Hz. Test pulse frequencies were then fixed at 0.05 Hz for the remaining of the experimental recording. The intensity used for the test pulses of the LTP experiments elicited population spikes of 0.5-3 mV (about 50% of the maximal response obtained with I/O curves) (Abraham et al., 1993; Steward et al., 1998).

LTP tetanization was performed as described (Steward et al., 1998). 400 Hz trains of 20 msec duration were delivered once every 10 sec. The individual pulses within the trains were of the same configuration as the test pulses, except for the intensities employed. Tetanization was delivered continuously for 120 or 180 min, depending on the individual experiment (2 hr and 3 hr time course, respectively). During delivery of the initial 400 Hz trains, the EEG was carefully monitored for any change indicating that an epileptic AD had occurred. None were ever observed in these experiments. Subsequent to tetanization, recordings were taken at baseline intensities for 30 min or more, whereupon the animals were left until perfused. Additionally, for some animals, recordings were taken for 5-10 min immediately prior to perfusion. Animals were perfusion-fixed 2-3 hours after delivery of the first train. Weight- and gender-matched control animals were anesthetized and processed in parallel.

Hippocampal ADs were evoked using 1 msec biphasic pulses delivered in a 60 Hz train for an initial duration of 1 sec (Cain et al., 1992). If an AD was not elicited, the duration of the train was increased, or the intensity was increased and the train delivered again after several minutes. Tetanization was repeated in this manner until an AD of at least 10 sec duration was elicited and recorded from the EEG. Using this approach, we produced either a single AD of at least 10 sec duration or, typically, two ADs in which case only the second one was of at least 10 sec duration. The duration of recorded hippocampal ADs was typically between 10 and 30 sec. Animals were perfusion-fixed 2-3 hours after induction of an AD. Weight- and gender-matched control animals were anesthetized and processed in parallel.

Preparation of Specimens. Cardiac perfusion was performed with 150 ml freshly prepared 4% formaldehyde (made from paraformaldehyde) in phosphate-buffered saline (PBS; 13.7 mM NaCl, 0.27 mM KCl, 0.43 mM Na₂HPO₄, 0.14 mM KH₂PO₄, pH 7.4). Brains were placed in ice-cold formaldehyde solution overnight, transferred successively to 12%, 16% and 20% sucrose solution, and embedded in Tissue-Tek (Sakura Finetek USA, Torrance, Calif.). Specimens were then cryosectioned onto microscope slides (Fisher, Pittsburgh, Pa.) (Lin et al., 2001). Al tissue sections used for this work were from equivalent caudo-rostral positions, corresponding to plate number 34-36 in the atlas of Paxinos and Watson (1998). In particular, to ensure comparability, sections from stimulated brains were chosen from a narrow area in the immediate vicinity of the stimulating electrode.

In Situ Hybridization and Immunocytochemistry. RNA probes against BC1 RNA were generated from plasmid pMK1 (Tiedge, 1991; Tiedge et al., 1991). Probes specific for Arc mRNA were generated from a clone containing a 3.032 kb cDNA insert (Lyford et al., 1995). This plasmid contains coding region, 3′ UTR and part of the 5′ UTR. Arc mRNA was used as a positive control in all experiments. ³⁵S-labeled RNA probes were transcribed from linearized templates, using T3 or T7 RNA polymerase as recommended by the manufacturer (Roche Diagnostics Corporation, Indianapolis, Ind.). Prehybridization and hybridization steps were carried out as described (Tiedge, 1991). High stringency washes were performed at 50° C.

For immunocytochemistry, sections were refixed in 4% formaldehyde/PBS directly after thawing, and then washed in PBS for 15 min. Unspecific binding was blocked with 5% BSA in PBS for 15 min. Sections were incubated with anti-synaptophysin monoclonal antibody 7.2 (Sigma, St. Louis, Mo.) for 24 hours at 4° C. (1:200 dilution in PBS). A biotinylated secondary antibody (anti-mouse IgG; Amersham Biosciences, Piscataway, N.J.) was applied for 2 hours (1:100 dilution) and decorated with a streptavidine-rhodamine conjugate (Molecular Probes, Eugene, Oreg.). Between all steps, sections were washed in PBS for 30 min. Sections were mounted in glycerol and immediately examined by fluorescence microscopy. To prevent drying out of tissue sections, all procedures were performed in a humid-atmosphere box. Control sections were processed the same way except that the primary antibody mixture was replaced by PBS.

Emulsion Autoradiography. Emulsion autoradiography was performed as previously described (Tiedge, 1991). In brief, dried sections were dipped in NTB2 emulsion (Eastman Kodak, Rochester, N.Y.) diluted 1:1 with HPLC-grade water, air dried, and exposed at 4° C. for 3 days (BC1 RNA) or 7 days (Arc mRNA). After photographic development (D-19 developer, 50% strength, and Rapid-Fix; Eastman Kodak), sections were stained with cresyl violet, dehydrated, and mounted in DPX (Fluka, Ronlconkoma, N.Y.).

Quantitative Analysis. Sections were analyzed and photographed on a Nikon Microphot-FXA microscope (Nikon, Melville, N.Y.), using dark field or epifluorescence optics. X-ray autoradiograms were either analyzed with the Nikon Microphot or with an Nikon Diaphot 300 inverted microscope. Images were acquired with a SONY DKC-5000 3CCD camera Photoshop software (Adobe Systems, San Jose, Calif.) was used to measure expression levels as described (Lehr et al., 1997; Lehr et al., 1999). For quantitative analysis of autoradiograms, regions of interest (ROIs) were selected in CA3 (stratum radiatum), CA1 (stratum radiatum and pyramidale), and dentate gyrus (stratum moleculare). Optical densities in ROIs were calculated from measured luminosity values using Lambert-Beer's law. To identify activity-dependent changes in RNA expression, ipsi- and contralateral sides were measured separately for all 3 ROIs and were plotted as ratios of signal intensities (ipsi/contra). Because of animal-to-animal variation of the hybridization signal (and, to a lesser degree, and section-to-section variation within the same animal), we restricted all quantitative analyses to comparisons within the same section. For quantitative analysis of autoradiographic silver grains, ROIs in each of stratum radiatum and stratum pyramidale were selected, and signal intensities in ROIs were calculated by subtracting background luminosity over glass from luminosity over ROI. To test for activity-dependent changes in subcellular RNA distribution, the values were plotted as ratios of radiatum/pyramidale for both stimulated and unstimulated hippocampi. Three to six coronal sections of the area of the mid-dorsal hippocampus were selected from each animal. Results were statistically evaluated by analysis of variance (one-way ANOVA) or by Student's t-test, using InStat software (www.rdg.ac.uk/ssc/instat/instat.htrnl; University of Reading, UK). In either case, level of significance was set at P<0.05.

### Example 4

### Results

It was the overall objective of this work to establish whether expression of the translational modulator BC1 RNA is itself subject to activity-dependent modulation. To address this question, we examined BC1 expression patterns after induction of LTP and after induction of epileptiform activity. In all experiments, Arc mRNA was probed as a positive control in the same respective animal as BC1 RNA.

Spatiotemporal BC1 Expression Patterns Are Not Significantly Altered Following Induction of LTP

We analyzed expression and localization of BC1 RNA during the protein synthesis-dependent phase of LTP in live animals. Rats were implanted with electrodes for stimulation of the left perforant path and for recording of field potentials in the ipsilateral dentate gyrus. Because high-frequency stimulation of the perforant path induces LTP in dentate granule cells as well as in pyramidal cells of CA3 and CA1 (Berger and Yeckel, 1991), we used recordings from the dentate gyrus as an index for LTP induction in all hippocampal regions.

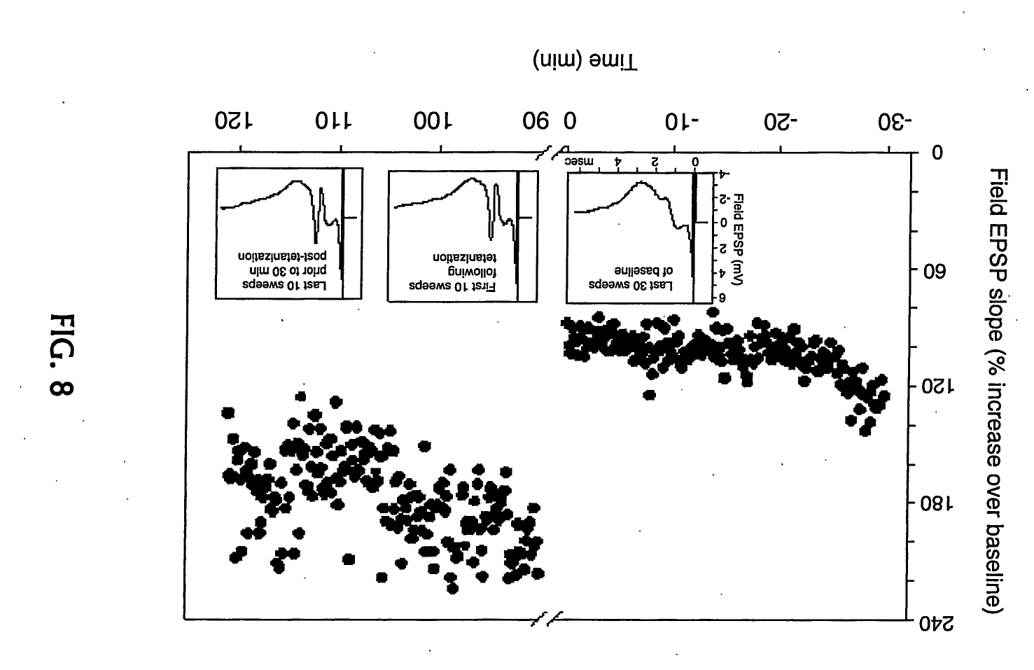

To induce LTP, stimulation was delivered for 90 min at an intensity that evoked a 0.5-3 mV population spike (PS). Physiological recordings confirmed that such stimulation induced LTP in every experiment. Unilateral high-frequency stimulation produced a robust potentiation of the field excitatory postsynaptic potential (fEPSP) slope and PS in the ipsilateral dentate gyrus. FIG. 8 shows the induction of LTP in a representative experiment. The fEPSP slope and PS clearly increased after high-frequency stimulation and remained elevated for the time of the recording (minimum of 30 min). Because even a short period of epileptiform activity can result in changes of RNA expression (see for example Isackson et al., 1991), we monitored the hippocampal EEG throughout all electrophysiological experiments. No ADs were observed during any of the LTP experiments, and none of the animals showed a depression of evoked responses after the high-frequency stimulation period that would indicate seizure activity.

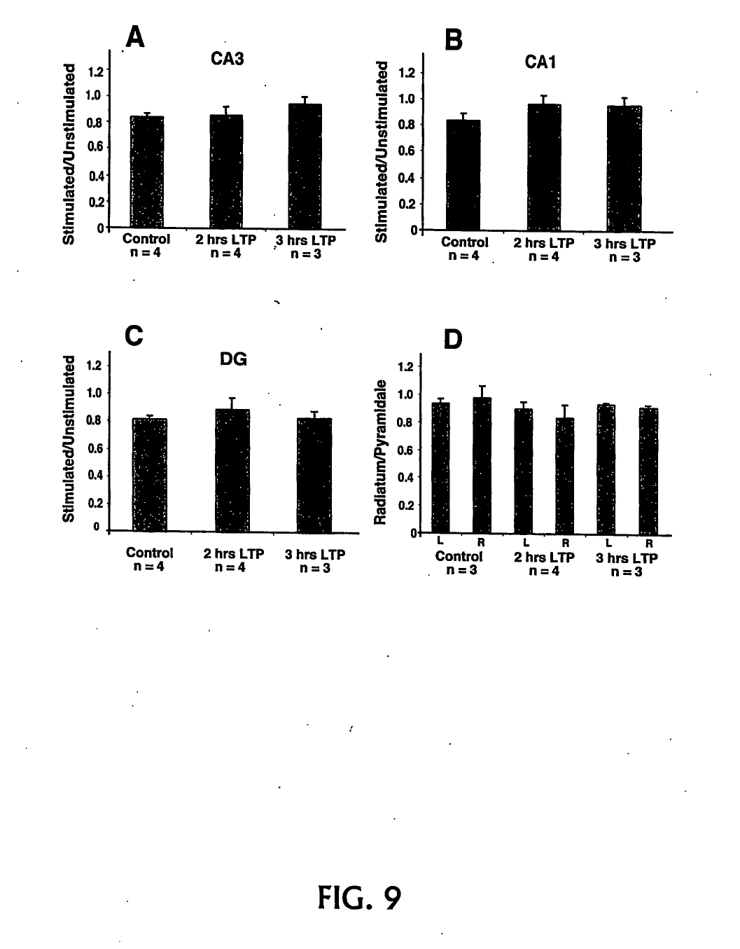

Brains of stimulated and control animals were analyzed for BC1 expression by in situ hybridization No appreciable changes were detected by visual inspection of any of the hippocampal areas. We analyzed brains 2 and 3 hours after stimulation and quantified BC1 expression in different regions of the hippocampal formation (FIG. 9A-C). Induction of LTP did not result in a significant change in BC1 expression levels in any of the analyzed areas. We also failed to observe significant alterations in ratios of BC1 expression in dendritic vs. somatic layers (FIG. 9D). Analogous results were obtained 1 and 4 hours after LTP induction (data not shown). To validate the adequacy of our stimulation paradigm, we analyzed the expression of Arc mRNA, an RNA that is known to be upregulated by LTP-inducing high-frequency stimulation (Link et al., 1995; Lyford et al., 1995). After high-frequency stimulation, this RNA was probed in brain sections adjacent to those probed for BC1 RNA. We found that Arc mRNA was strongly upregulated in cell bodies and dendrites of the stimulated dentate gyrus and remained so for several hours. This result confirms that our experimental design was suitable to generate and detect activity-dependent changes in RNA expression levels.

In summary, these results show that BC1 expression was not significantly altered during the protein-synthesis dependent phase of LTP. Thus, for LTP maintenance, modulation of BC1 expression levels appears not to be required in this experimental paradigm.

BC1 Expression Levels Are Downregulated Following Induction of Epileptiform Activity

Seizure events are generated by massive synaptic excitation and are accompanied by increased protein synthesis (Elmér et al., 1998; Wallace et al., 1998; Watkins et al., 1998; Koubi et al., 1999). To establish whether expression of translational repressor BC1 RNA is modulated under such conditions, we induced epileptiform activity in brains of live animals. Animals were implanted with electrodes to the right hippocampus for Schaffer collateral stimulation and recording of the hippocampal EEG. A 60 Hz kindling protocol was used to generate single hippocampal ADs of 10-30 sec duration (see Materials and Methods).

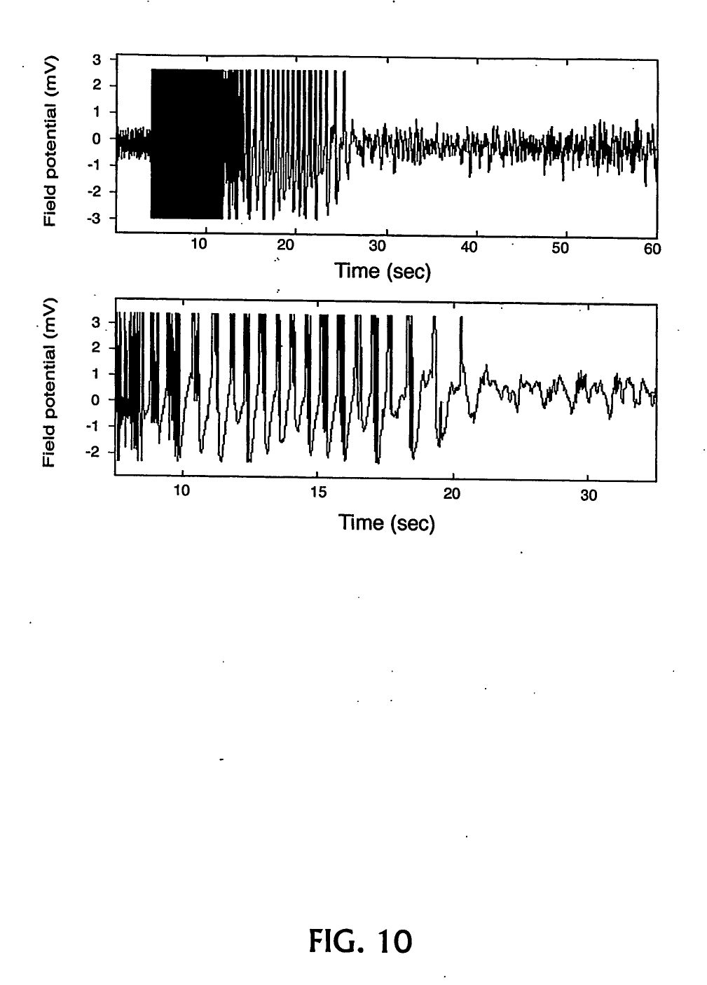

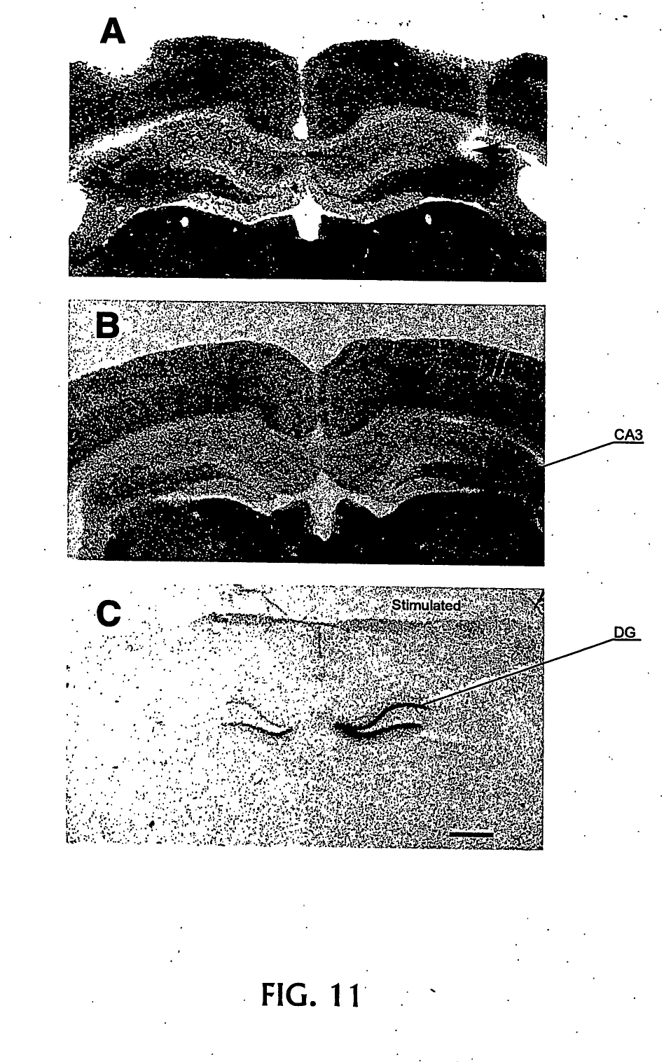

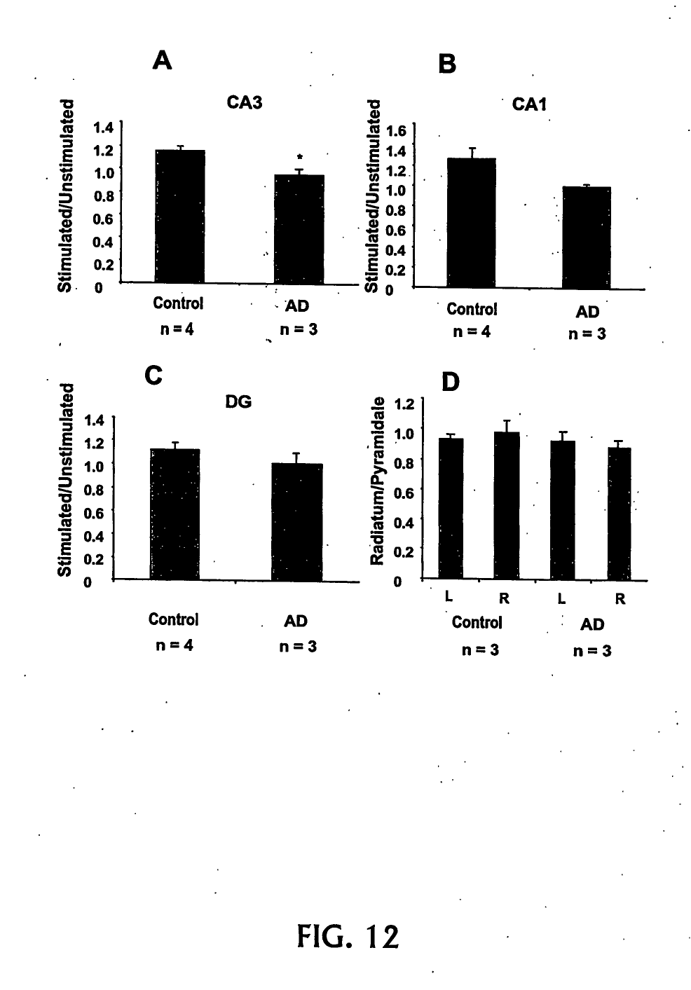

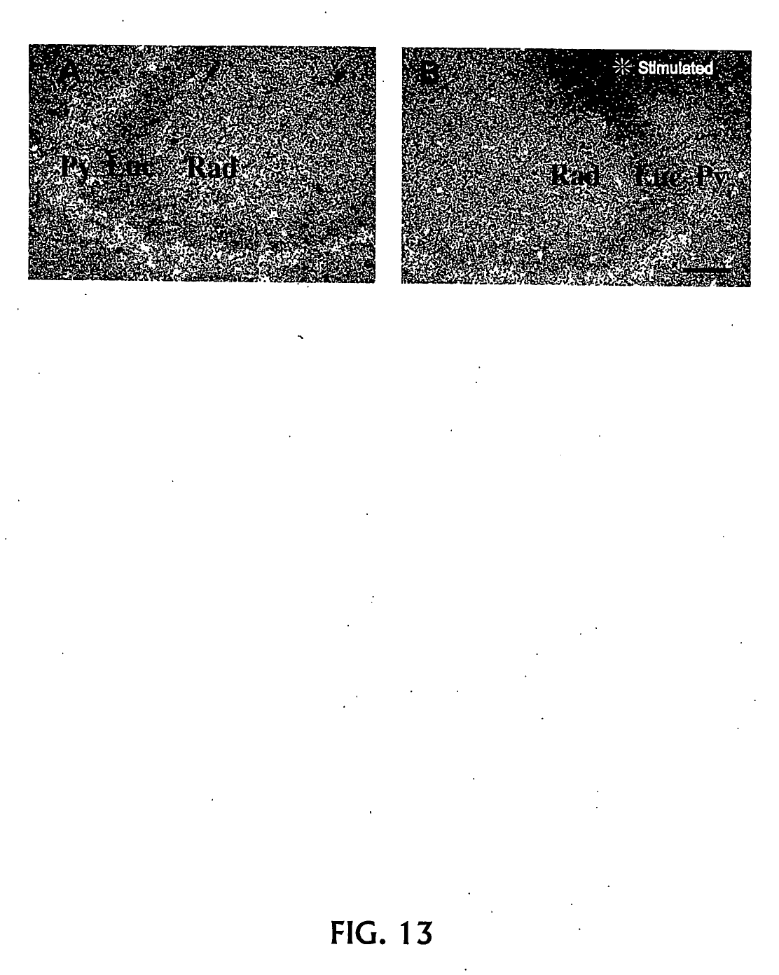

FIG. 10 shows the hippocampal EEG of a rat brain during a kindling-induced AD. Synchronized neural activity occurred shortly after high-frequency stimulation and revealed the typical pattern of an AD. This activation strongly induced the expression of Arc mRNA (Link et al., 1995; Lyford et al., 1995), used here for reference as a molecular positive control (FIG. 11C). The result indicates that induction of an epileptic discharge was sufficient to modulate expression of a dendritic RNA Autoradiograms in FIGS. 11A and 11B show the distribution of BC1 RNA after seizure induction, compared with that in an unstimulated control animal. In unstimulated animals (FIG. 1B), we consistently observed higher expression of BC1 RNA in the right hippocampus than in the left one. Such asymmetric expression may be due to differences in morphology, preferred usage of one hemisphere, or other left-right functional brain asymmetries that have previously been reported in various animal systems (Glick and Ross, 1981; Davidson and Hugdahl, 1994; Hobert et al., 2002; Toga and Thompson, 2003). Induction of epileptiform activity in the right hippocampus caused a marked decrease of the BC1 RNA signal on the ipsilateral side, resulting in now virtually identical expression levels in ipsi- and contralateral hippocampus (FIG. 11A). The change in BC1 expression was not confined to CA1 neurons but appeared throughout the ipsilateral hippocampus. Quantitative analysis revealed a significant decrease of BC1 expression levels in the CA3 field and a smaller decrease—one that did not reach statistical significance—in CA1 and in dentate gyrus (FIG. 12). It should be noted in this context that epileptiform events are typically not restricted to their seizure focus sites but propagate to surrounding tissue (McCormick and Contreras, 2001) where they can thus trigger changes in expression levels, as is the case here for Arc mRNA and BC1 RNA. Image analysis revealed no relative change in the spatial and laminar distribution of BC1 RNA in CA3 (FIGS. 12, 13), thus suggesting a uniform reduction in BC1 levels in both dendritic and cell body layers. This result indicates that levels of BC1 RNA were downregulated in a cell-wide fashion throughout principal CA3 neurons. Thus, induction of epileptiform activity resulted in a marked downregulation of somatodendritic BC1 RNA in the stimulated hippocampus, whereas—in the same area of the same animals—a control RNA (Arc mRNA) was upregulated.

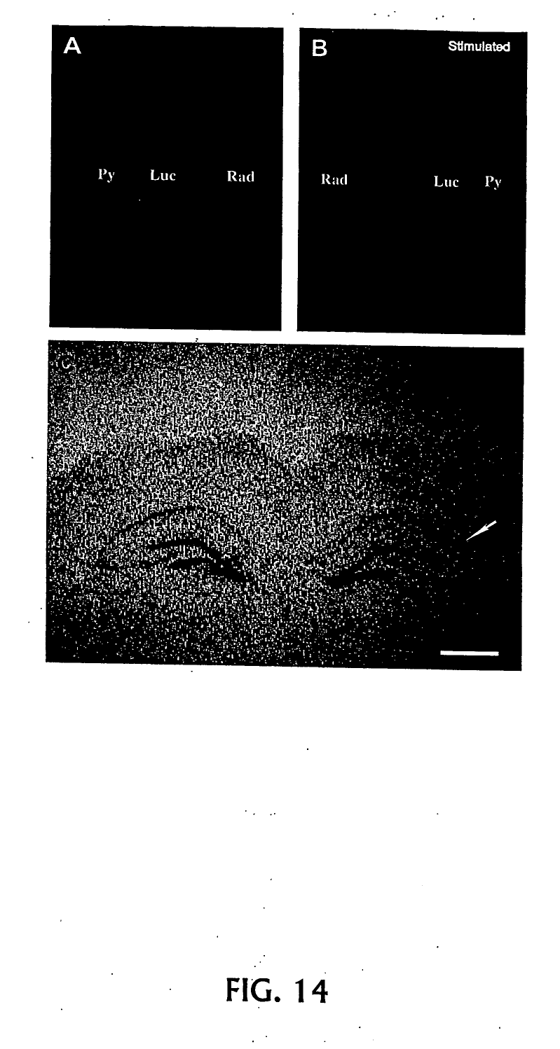

It can not formally be ruled out that the observed decrease was due to damage of hippocampal tissue or to a loss of innervation that could hypothetically have occurred subsequent to stimulation. To control for this possibility, we probed for the presence of mossy fiber terminals in seizured animals by using an antibody specific for synaptophysin, a marker for presynaptic specializations (Jahn et al., 1985). In immunofluorescence microscopy, such specializations are visualized as clusters of discrete labeling puncta (Fletcher et al., 1994). We observed that the density of synaptophysin labeling puncta in CA3 was comparable in both hemispheres of unilaterally kindled animals (FIG. 14). In fact, it appears that the synaptophysin labeling signal was somewhat stronger in stratum lucidum of the stimulated side (although no attempt was made to quantify this observation). The results confirm that innervation of CA3 pyramidal cells was not negatively affected following kindling-induced ADs. Cresyl violet staining also failed to reveal any signs of tissue deterioration We furthermore examined all seizured and control animals for expression of Arc mRNA. In all cases, Arc mRNA was significantly upregulated in the seizured hippocampus, thus confirming that gene expression mechanisms were not compromised in hippocampal neurons. While most prominent in the dentate gyrus, upregulation of Arc mnRNA was also observed in CA3 and CA1 after induction of strong seizures (FIG. 14C). These results provide further evidence that ADs easily spread from the original sites of induction. Significantly, moreover, the data clearly show that cell viability and functionality were not adversely affected by AD induction. The results therefore provide further confirmation that the observed downregulation of BC1 expression levels was specific and not the result of a general downregulation of gene expression.

Taken together, the data establish that BC1 expression is specifically and significantly reduced following induction of epileptiform activity. We conclude that BC1 RNA, itself a translational repressor, is subject to modulation by strong synaptic activation in vivo.

### Example 5

### BC1-mediated Translation Repression is Dependent on Simultaneous Functional Interactions with eIF4A and PABP

BC1 RNA represses translation initiation by targeting eIF4-mediated recruitment of the small ribosomal subunit to the mRNA, a key step in eukaryotic initiation that is dependent on the eIF4 group of factors and is stimulated by PABP. As demonstrated in Example 2, BC1 RNA binds to eIF4A and PABP. In this example, the question of whether such direct physical interactions form the basis for the functional role of BC1 RNA as a repressor of translation, was examined. The question was addressed by asking if BC1-repressed translation could be ‘rescued’ by back-titration with eIF4A or PABP, or stoichiometric combinations thereof.

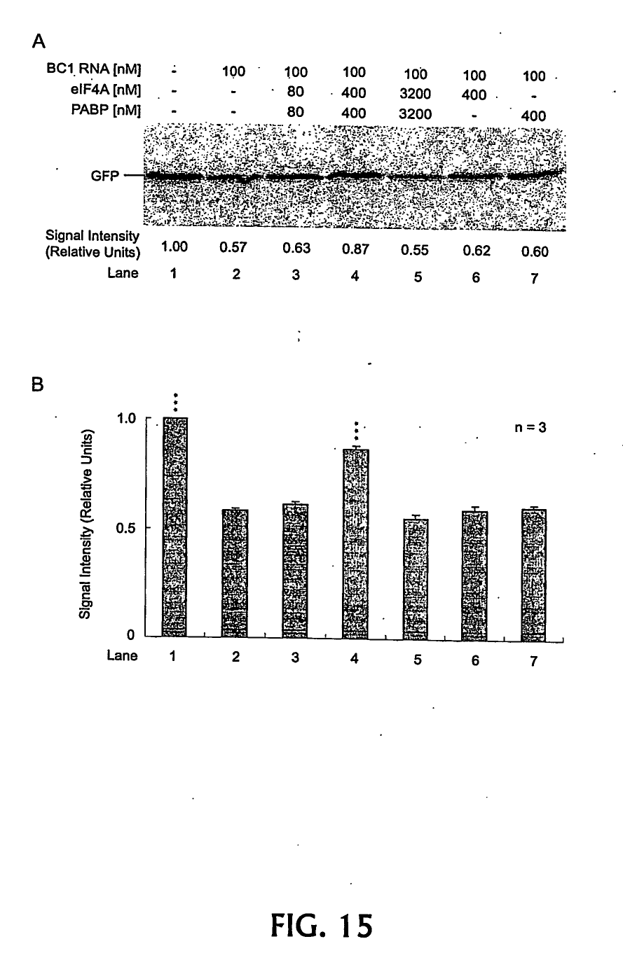

An IRES-mediated initiation mode was chosen for these experiments, performed in the rabbit reticulocyte (RRL) cell-free translation system. Translation was programmed with green fluorescent protein (GFP) mRNA and was initiated from an IRES of the encephalomyocarditis (EMCV) subtype. Initiation from the EMCV IRES requires all initiation factors of the eIF4 family except cap-binding protein eIF4E. This initiation mode was demonstrated in Example 2 to be particularly sensitive to BC1-mediated repression. BC1 RNA effectively inhibited translation initiated on the EMCV IRES (50% repression at 100 nM BC1 RNA; FIG. 15). Titration with eIF4A resulted in a small increase in translational efficiency; however, throughout the concentration range tested (80-3200 nM), this increase failed to reach statistical significance (FIG. 15; 400 nM eIF4A is shown). Similarly, a small but insignificant rescue of translation was observed upon back-titration with PABP (FIG. 15). In clear contrast, however, BC1-repressed translation could be rescued by simultaneous, stoichiometric titration with eIF4A and PABP (FIG. 15). At 400 nM of both factors, translational efficiency was restored to almost 90% of standard (i.e. not BC1-repressed) levels. Rescue of BC1-repressed translation by eIF4A and PABP was effective only in a submicromolar concentration window as ‘over-titration’ failed to restore translational efficiency.

The above results directly support the notion that the molecular basis for BC1-mediated translational repression is a dual, simultaneous interaction with eIF4A and PABP. Interaction with only one of the two factors appears to be functionally insufficient as translation should in that case be restorable by back-titration with that factor alone. The data indicate that BC1 RNA interacts with both factors at the same time, presumably as they are contained in a complex.

### REFERENCES

Aakalu G, Smith W B, Nguyen N, Jiang C, Schuman E M (2001) Dynamic visualization of local protein synthesis in hippocampal neurons. Neuron 30:489-502.
Abraham W C, Mason S E, Demmer J, Williams J M, Richardson C L, Tate W P, Lawlor P A, Dragunow M (1993) Correlations between immediate early gene induction and the persistence of long-term potentiation Neuroscience 56:717-727.
Agrawal et al. (1995 Agrawal et al. (1995) Absorption, tissue distribution and in vivo stability in rats of a hybrid antisense oligonucleotide following oral administration. Biochem. Pharmacol. 50:571-576.
Banks, William, A., et al. (2002) Delivery across the blood-brain barrier of antisense directed against amyloid β:reversal of leaming and memory deficits in mice overpexpressing amyloid precursor protein. J. Pharmacol. and experimental therapeutics 297:1113-121.
Berger T W, Yeckel M F (1991) Long-term potentiation of entorhinal afferents to the hippocampus: enhanced propagation of activity through the trisynaptic pathway. In:

Long-term potentiation: A debate of current issues (Baudry M, Davis J L, eds), pp 327-356. Cambridge, Mass.: MIT Press.
Bianchi R, Wong R K (1995) Excitatory synaptic potentials dependent on metabotropic glutamate receptor activation in guinea-pig hippocampal pyramidal cells. J Physiol 487:663-676.
Binder D K, Croll S D, Gall C M, Scharfman H E (2001) BDNF and epilepsy: too much of a good thing? Trends Neurosci 24:47-53.
Bliss T W, Lomo T (1973) Long-lasting potentiation of synaptic transmission in the dentate area of the anaesthetized rabbit following stimulation of the perforant path. J Physiol 232:331-356.
Brosius J, Tiedge H (1995) Neural BC1 RNA: dendritic localization and transport In: Localized RNAs (Lipshitz HD, ed), pp 289-300. Austin: R G. Landes.
Brosius J, Tiedge H (1996) Reverse transcriptase—mediator of genomic plasticity. Virus Genes 11:163-179.
Brosius J, Tiedge H (2001) Dendritic BC1 RNA: intracellular transport and activity-dependent expression. In: Cell polarity and subcellular RNA localization (Richter D, ed), pp 129-138. Berlin: Springer.
Burgin K E, Waxham M N, Rickling S, Westgate S A, Mobley W C, Kelly P T (990) In situ hybridization histochemistry of Ca²⁺/calmodulin-dependent protein kinase in developing rat brain. J Neurosci 10:1788-1798.
Cain D P, Boon F, Hargreaves E L (1992) Evidence for different neurochemical contributions to long-term potentiation and to kindling and kindling-induced potentiation: role of NMDA and urethane-sensitive mechanisms. Exp Neurol 116:330-338.
Cao Q, Richter J D (2002) Dissolution of the maskin-eIF4E complex by cytoplasmic polyadenylation and poly(A)-binding protein controls cyclin B1 mRNA translation and oocyte maturation. EMBO J 21:3852-3862.
Cheng J-G, Tiedge H, Brosius J (1996) Identification and characterization of BC1 RNP particles. DNA Cell Biol 15:549-559.
Chervin R D, Pierce P A, Connors B W (1988) Periodicity and directionality in the propagation of epileptiform discharges across neocortex. J Neurophysiol 60:1695-1713.
Chicurel M E, Terrian D M, Potter H (1993) mRNA at the synapse: analysis of a synaptosomal preparation enriched in hippocampal dendritic spines. J Neurosci 13:4054-4063.
Crino P B, Eberwine J (1996) Molecular characterization of the dendritic growth cone: Regulated mRNA transport and local protein synthesis. Neuron 17:1173-1187.
Davidson R J, Hugdahl K (1994) Brain asymmetry. Cambridge, Massachusetts: MIT Press.
Davis H P, Squire L R (1984) Protein synthesis and memory: a review. Psychol Bull 96:518-559.
Dever T E (2002) Gene-specific regulation by general translation factors. Cell 108:545-556.
Eberwine J, Miyashiro K, Kachannina J E, Job C (2001) Local translation of classes of mRNAs that are targeted to neuronal dendrites. Proc Natl Acad Sci USA 98:7080-7085.
Eddy S R (2002) Computational genornics of noncoding RNA genes. Cell 109:137-140.
Elmér M, Kokaia Z, Kokaia M, Carnahan J, Nawa H, Lindvall O (1998) Dynamic changes of brain-derived neurotrophic factor protein levels in the rat forebrain after single and recurring kindling-induced seizures. Neuroscience 83:351-362.
Fletcher T L, Cameron P, De Camilli P, Banlcer G (1994) The distribution of synapsin I and synaptophysin in hippocampal neurons developing in culture. J Neurosci 11:1617-1626.
Fletcher T L, De Camilli P, Banker G (1994) Synaptogenesis in hippocampal cultures: evidence indicating that axons and dendrites become competent to form synapses at different stages of neuronal development. J Neurosci 14:6695-6706.
Frey U, Krug M, Reymann K G, Matthies H (1988) Anisomycin, an inhibitor of protein synthesis, blocks late phase of LTP phenomena in the hippocampal CA1 region in vitro. Brain Res 452:57-65.
Gardiol A, Racca C, Triller A (1999) Dendritic and postsynaptic protein synthetic machinery. J Neurosci 19:168-179.
Garner C C, Tucker R P, Matus A (1988) Selective localization of messenger RNA for cytoskeletal protein MAP2 in dendrites. Nature 336:674-677.
Geschwind N, Levitsky W (1968) Human brain: left-right asymmetries in temporal speech region. Science 161:186-187.
Gingras A-C, Raught B, Sonenberg N (1999) eIF4 initiation factors: effectors of mRNA recruitment to ribosomes and regulators of translation. Annu Rev Biochem 68:913-963.
Glick S D, Ross D A (1981) Lateralization of function in the rat brain. Trends Neurosci 4:196-199.
Goto Y, Araki T, Fukui M (1994) Propagation of hippocampal seizure activity arising from the hippocampus: a local cerebral blood flow study. Brain Res 634:203-213.
Gray N K, Hentze M W (1994) Iron regulatory protein prevents binding Qf the 43S translation pre-initiation complex to ferritin and eALAS mRNAs. EMBO J 13:3882-3891.
Greenough W T, Klintsova A Y, Irwin S A, Galvez R, Bates K E, Weiler I J (2001) Synaptic regulation of protein synthesis and the fragile X protein. Proc Natl Acad Sci USA 98:7101-7106.
Gu W, Hecht N R (1996) Translation of a testis-specific Cu/Zn superoxide dismutase (SOD-1) mRNA is regulated by a 65-kilodalton protein which binds to its 5′ untranslated region. Mol Cell Biol 16:4535-4543.
Hall, S. J., et al. (1997) The promise and reality of cancer gene therapy. Am. J. Hum. Genetics, 61:785-789.
Hausner T P, Giglio L M, Weiner A M (1990) Evidence for base-pairing between mammalian U2 and U6 small nuclear ribonucleoprotein particles. Genes Dev 4:2146-2156.
Hellen C U, Sarnow P (2001) Internal ribosome entry sites in eukaryotic mRNA molecules. Genes Dev 15:1593-1612.
Hershey J W B, Merrick W C (2000) The pathway and mechanism of initiation of protein synthesis. In: Translational control of gene expression (Sonenberg N, Hershey J W B, Mathews M B, eds), pp 33-88. Cold Spring Harbor: Cold Spring Harbor Laboratory Press.
Hobert O, Johnston R J, Chang S (2002) Left-right asymmetry in the nervous system: the Caenorhabditis elegans model. Nat Rev Neurosci 3:629-640.
Imataka H, Gradi A, Sonenberg N (1998) A newly identified N-terminal amino acid sequence of human eIF4G binds poly(A)-binding protein and functions in poly(A)-dependent translation. EMBO J 17:7480-7489.
Isackson P J, Huntsman M M, Murray K D, Gall C M (1991) BDNF mRNA expression is increased in adult rat forebrain after limbic seizures: temporal patterns of induction distinct from NGF. Neuron 6:937-948.
Jackson R J (2000) A comparative view of initiation site selection mechanisms. In: Translational control of gene expression (Sonenberg N, Hershey J W B, Mathews M B, eds), pp 127-183. Cold Spring Harbor: Cold Spring Harbor Laboratory Press.
Jahn R, Schiebler W, Ouimet C, Greengard P (1985) A 38,000-dalton membrane protein (p38) present in synaptic vesicles. Proc Natl Acad Sci USA 82:4137-4141.
Job C, Eberwine J (2001a) Identification of sites for exponential translation in living dendrites. Proc Natl Acad Sci USA 98:13037-13042.
Job C, Eberwine J (2001b) Localization and translation of mRNA in dendrites and axons. Nat Rev Neurosci 2:889-898.
Iacharmina J E, Job C, Crino P, Eberwine J (2000) Stimulation of glutamate receptor protein synthesis and membrane insertion within isolated neuronal dendrites. Proc Natl Acad Sci USA 97:11545-11550.
Khaleghpour K, Kahvejian A, De Crescenzo G, Roy G, Svitkin Y V, Imataka H, O'Connor-McCourt M, Sonenberg N (2001) Dual interactions of the translational repressor Paip2 with poly(A) binding protein. Mol Cell Biol 21:5200-5213.
Kiebler M A, DesGroseillers L (2000) Molecular insights into mRNA transport and local translation in the mammalian nervous system. Neuron 25:19-28.
Kindler S, Mohr E, Richter D (1997) Quo vadis: extrasomatic targeting of neuronal mRNAs in mammals. Mol Cell Endocrinol 128:7-10.
Koubi D, Gharib A, Gagnon J, Andrieu J P, Bobillier P, Sarda N (1999) Early and prolonged widespread increase in brain protein synthesis following a single electroconvulsive shock in free-moving rats. Brain Res 821:111-116.
Krug M, Lossner B, Ott T (1984) Anisomycin blocks the late phase of long-term potentiation in the dentate gyrus of freely moving rats. Brain Res Bull 13:39-42.
Kuhl D, Skehel P (1998) Dendritic localization of mRNAs. Curr Opin Neurobiol 8:600-606.
Kuryshev V Y, Skryabin B V, Kremerskothen J, Jurka J, Brosius J (2001) Birth of a gene: locus of neuronal BC200 snmRNA in three prosimians and human BC200 pseudogenes as archives of change in the Anthropoidea lineage. J Mol Biol 309:1049-1066.
Kwon S, Barbarese E, Carson J H (1999) The cis-acting RNA trafficking signal from myelin basic protein mRNA and its cognate trans-acting ligand hnRNP A2 enhance cap-dependent translation. J Cell Biol 147:247-256.
Le H, Tanguay R L, Balasta M L, Wei C C, Browning K S, Metz A M, Goss D J, Gallie D R (1997) Translation initiation factors eIF-iso4G and eIF-4B interact with the poly(A)-binding protein and increase its RNA binding activity. J Biol Chem 272:16247-16255.
Lehr H A, Mankoff D A, Corwin D, Santeusanio G, Gown A M (1997) Application of photoshop-based image analysis to quantification of hormone receptor expression in breast cancer. J Histochem Cytochem 45:1559-1565.
Lehr H A, van der Loos C M, Teeling P, Gown A M (1999) Complete chromogen separation and analysis in double immunohistochemical stains using Photoshop-based image analysis. J Histochem Cytochem 47:119-126.
LeMay M (1982) Morphological aspects of human brain asymmetry. Trends Neurosci 5:273-275.
Lin Y, Brosius J, Tiedge H (2001) Neuronal BC1 RNA: co-expression with growth-associated protein-43 messenger RNA Neuroscience 103:465-479.
Link W, Konietzko U, Kauselmann G, Krug M, Schwanke B, Frey U, Kuhl D (1995) Somatodendritic expression of an immediate early gene is regulated by synaptic activity. Proc Natl Acad Sci USA 92:5734-5738.
Liu H M, Lei D L, Yang D L (1996) Kainate-induced brain lesion: similar local and remote histopathological and molecular changes as in ischemic brain infarct. J Neuropathol Exp Neurol 55:787-794.
Lomakin I B, Hellen C U, Pestova T V (2000) Physical association of eukaryotic initiation factor 4G (eIF4G) with eIF4A strongly enhances binding of eIF4G to the internal ribosomal entry site of encephalomyocarditis virus and is required for internal initiation of translation. Mol Cell Biol 20:6019-6029.
Lyford G L, Yamagata K, Kaufmann W E, Barnes C A, Sanders L K, Copeland N G, Gilbert D J, Jenkins N A, Lanahan A A, Worley P F (1995) Arc, a growth factor and activity-regulated gene, encodes a novel cytoskeleton-associated protein that is enriched in neuronal dendrites. Neuron 14:433-345.
Malenka R C, Nicoll R A (1999) Long-term potentiation—a decade of progress? Science 285:1870-1874.
McCormick D A, Contreras D (2001) On the cellular and network bases of epileptic seizures. Annu Rev Physiol 63:815-846.
Merlin L R, Bergold P J, Wong R K (1998) Requirement of protein synthesis for group I mGluR-mediated induction of epileptiform discharges. J Neurophysiol 80:989-993.
Michel Y M, Borman A M, Paulous S, Kean K M (2001) Eukaryotic initiation factor 4G-poly(A) binding protein interaction is required for poly(A) tail-mediated stimulation of picomavirus internal ribosome entry segment-driven translation but not for X-mediated stimulation of hepatitis C virus translation. Mol Cell Biol 21:4097-4109.
Miles R, Wong R K (1983) Single neurones can initiate synchronized population discharge in the hippocampus. Nature 306:371-373.
Miles R, Wong R K (1987) Latent synaptic pathways revealed after tetanic stimulation in the hippocampus. Nature 329:724-726.
Miles R, Wong R K, Traub R D (1984) Synchronized afterdischarges in the hippocampus: contribution of local synaptic interactions. Neuroscience 12:1179-1189.
Muddashetty R, Khanam T, Kondrashov A, Bundman M, Iacoangeli A, Kremerskothen J, Duning K, Barnekow A, Hüttenhofer A, Tiedge H, Brosius J (2002) Poly(A)-binding protein is associated with neuronal BC1 and BC200 ribonucleoprotein particles. J Mol Biol 321:433-445.
Muslimov I A, Banker G, Brosius J, Tiedge H (1998) Activity-dependent regulation of dendritic BC1 RNA in hippocampal neurons in culture. J Cell Biol 141:1601-1611.
Muslimov I A, Santi E, Homel P, Perini S, Higgins D, Tiedge H (1997) RNA transport in dendrites: a cis-acting targeting element is contained within neuronal BC1 RNA J Neurosci 17:4722-4733.
Osten P, Valsamis L, Harris A, Sacktor T C (1996) Protein synthesis-dependent formation of protein kinase Mz in long-term potentiation. J Neurosci 16:2444-2451.
Otani S, Abraham W C (1989) Inhibition of protein synthesis in the dentate gyrus, but not the entorhinal cortex, bloclcs maintenance of long-term potentiation in rats. Neurosci Lett 106:175-180.
Otero L J, Ashe M P, Sachs A B (1999) The yeast poly(A)-binding protein PabIp stimulates in vitro poly(A)-dependent and cap-dependent translation by distinct mechanisms. EMBO J 18:3153-3163.
Pause A, MethotN, Svitkin Y, Merrick W C, Sonenberg N (1994) Dominant negative mutants of mammalian translation initiation factor eIF-4A define a critical role for eIF-4F in cap-dependent and cap-independent initiation of translation. EMBO J 13:1205-1215.
Paxinos G, Watson C (1998) The rat brain in stereotaxic coordinates. San Diego: Academic press.
Pestova T V, Hellen C U, Shatsky I N (1996a) Canonical eukaryotic initiation factors determine initiation of translation by internal ribosomal entry. Mol Cell Biol 16:6859-6869.
Pestova T V, Kolupaeva V G, Lomakin I B, Pilipenko E V, Shatsky I N, Agol V I, Hellen C U (2001) Molecular mechanisms of translation initiation in eukaryotes. Proc Natl Acad Sci USA 98:7029-7036.
Pestova T V, Shatsky I N, Fletcher S P, Jackson R J, Hellen C U (1998) A prokaryotic-like mode of cytoplasmic eukaryotic ribosome binding to the initiation codon during internal translation initiation of hepatitis C and classical swine fever virus RNAs. Genes Dev 12:67-83.
Pestova T V, Shatsky I N, Hellen C U (1996b) Functional dissection of eukaryotic initiation factor 4F: the 4A subunit and the central domain of the 4G subunit are sufficient to mediate internal entry of 43S preinitiation complexes. Mol Cell Biol 16:6870-6878.
Pinkstaff J K, Chappell S A, Mauro V P, Edelman G M, Krushel L A (2001) Internal initiation of translation of five dendritically localized neuronal mRNAs. Proc Natl Acad Sci USA 98:2770-2775.
Raught B, Gingras A-C, Gygi S P, Imataka H, Morino S, Gradi A, Aebersold R, Sonenberg N (2000) Serum-stimulated, rapamycin-sensitive phosphorylation sites in the eukaryotic translation initiation factor 4GI. EMBO J 19:434-444.
Raught B, Gingras A-C, Sonenberg N (2001) The target of rapamycin (TOR) proteins. Proc Natl Acad Sci USA 98:7037-7044.
Richter D, ed (2001) Cell polarity and subcellular RNA localization. Berlin: Springer.
Rozhdestvensky T, Kopylov A, Brosius J, Hüttenhofer A (2001) Neuronal BC1 RNA structure: evolutionary conversion of a tRNA^{Ala} domain into an extended stem-loop structure. RNA 7:1-9.
Sauter, Bernhard V. et al. (2000) Adenovirus-mediated gene transfer of endostatin in vivo results in high level of transgene expression and inhibition of tumor growth and metastases. Proc. Natl. Acad. Sci. USA 97(9):4802-4807.
Sachs A (2000) Physical and functional interactions between the mRNA cap structure and the poly(A) tail. In: Translational control of gene expression (Sonenberg N, Hershey J W B, Mathews M B, eds), pp 447-465. Cold Spring Harbor: Cold Spring Harbor Laboratory Press.
Schacher S, Wu F (2002) Synapse formation in the absence of cell bodies requires protein synthesis. J Neurosci 22:1831-1839.
Scheetz A J, Nairn A C, Constantine-Paton M (2000) NMDA receptor-mediated control of protein synthesis at developing synapses. Nat Neurosci 3:211-216.
Sloviter R S (1999) Status epilepticus-induced neuronal injury and network reorganization. Epilepsia 40:34-41.
Smith D B, Johnson K S (1988) Single-step purification of polypeptides expressed in Escherichia coli as fusions with glutathione S-transferase. Gene 67:31-40.
Steward O, Levy W B (1982) Preferential localization of polyribosomes under the base of dendritic spines in granule cells of the dentate gyrus. J Neurosci 2:284-291.
Steward O, Halpain S (1999) Lamina-specific synaptic activation causes domain-specific alterations in dendritic immunostaining for MAP2 and CAM kinase II. J Neurosci 19:7834-7845.
Steward O, Schuman E M (2001) Protein synthesis at synaptic sites on dendrites. Annu Rev Neurosci 24:299-325.
Steward O, Wallace C S, Lyford G L, Worley P F (1998) Synaptic activation causes the mRNA for the IEG Arc to localize selectively near activated postsynaptic sites on dendrites. Neuron 21:741-751.
Storz G (2002) An expanding universe of noncoding RNAs. Science 296:1260-1263.
Sutula T, He X X, Cavazos J, Scott G (1988) Synaptic reorganization in the hippocampus induced by abnormal functional activity. Science 239:1147-1150.
Sutula T, Pitkänen A, eds (2002) Do seizures damage the brain. Progress in Brain Research. Amsterdam, Elsevier.
Svitkin Y V, Imataka H, Khaleghpour K, Kahvejian A, Liebig H D, Sonenberg N (2001) Poly(A)-binding protein interaction with eIF4G stimulates picomavirus IRES-dependent translation. RNA 7:1743-1752.
Tang S J, Reis G, Kang H, Gingras A-C, Sonenberg N, Schuman E M (2002) A rapamycin-sensitive signaling pathway contributes to long-term synaptic plasticity in the hippocampus. Proc Natl Acad Sci USA 99:467-472.
Tarun S Z, Jr., Sachs A B (1996) Association of the yeast poly(A) tail binding protein with translation initiation factor eIF-4G. EMBO J 15:7168-7177.
Thomson A M, Rogers J T, Walker C E, Staton J M, Leedman P J (1999) Optimized RNA gel-shift and UV cross-linking assays for characterization of cytoplasmic RNA-protein interactions. BioTechniques 27:1032-1042.
Tiedge H (1991) The use of UV light as a cross-linking agent for cells and tissue sections in in situ hybridization. DNA Cell Biol 10:143-147.
Tiedge H, Bloom F E, Richter D (1999) RNA, Whither Goest Thou? Science 283:186-187.
Tiedge H, Brosius J (1996) Translational machinery in dendrites of hippocampal neurons in culture. J Neurosci 16:7171-7181.
Tiedge H. Chen W, Brosius J (1993) Primary structure, neural-specific expression, and dendritic location of human BC200 RNA. J Neurosci 13:2382-2390.
Tiedge H, Fremeau R T, Jr., Weinstock P H, Arancio O, Brosius J (1991) Dendritic location of neural BC1 RNA Proc Natl Acad Sci USA 88:2093-2097.
Toga A W, Thompson P M (2003) Mapping brain asymmetry. Nat Rev Neurosci 4:3748.
Tooyama I, Bellier J P, Park M, Minnasch P, Uemura S, Hisano T, Iwami M, Aimi Y, Yasuhara O, Kimura H (2002) Morphologic study of neuronal death, glial activation, and progenitor cell division in the hippocampus of rat models of epilepsy. Epilepsia 43:3943.
Torre E R, Steward O (1992) Demonstration of local protein synthesis within dendrites using a new cell culture system which permits the isolation of living axons and dendrites from their cell bodies. J Neurosci 12:762-772.
Torre E R, Steward O (1996) Protein synthesis within dendrites: glycosylation of newly synthesized proteins in dendrites of hippocampal neurons in culture. J Neurosci 16:5967-5978.
Wakyama M, Imataka H, Sonenberg N (2000) Interaction of eIF4G with poly(A)-binding protein stimulates translation and is critical for Xenopus oocyte maturation. Curr Biol 10:1147-1150.
Wallace C S, Lyford G L, Worley P F, Steward O (1998) Differential intracellular sorting of immediate early gene mRNAs depends on signals in the mRNA sequence. J Neurosci 18:26-35.
Wang H, Iacoangeli A, Popp S, Muslimov I A, Imataka H, Sonnenberg N, Lomakin I B, Tiedge H (2002) Dendritic BC1 RNA: functional role in regulation of translation initiation. J Neurosci 22:10232-10241.
Wang W, Brunet F G, Nevo E, Long M (2002) Origin of sphinx, a young chimeric RNA gene in Drosophila melanogaster. Proc Natl Acad Sci USA 99:4448-4453.
Watkins C J, Pei Q, Newberry N R (1998) Differential effects of electroconvulsive shock on the glutamate receptor mRNAs for NR2A, NR2B and mGluR5b. Mol Brain Res 61:108-113.
Wells D G, Dong X, Quinlan E M, Huang Y S, Bear M F, Richter J D, Fallon J R (2001) A role for the cytoplasmic polyadenylation element in NMDA receptor-regulated mRNA translation in neurons. J Neurosci 21:9541-9548.
Wells D G, Richter J D, Fallon J R (2000) Molecular mechanisms for activity-regulated protein synthesis in the synapto-dendritic compartment. Curr Opin Neurobiol 10:132-137.
West N, Roy-Engel A, Imataka H, Sonenberg N, Deininger P (2002) Shared Protein Components of SINE RNPs. J Mol Biol 321:423-432.
Wong R K, Traub R D (1983) Synchronized burst discharge in disinhibited hippocampal slice. I. Initiation in CA2-CA3 region. J Neurophysiol 49:442-458.
Wong R K, Traub R D, Miles R (1986) Cellular basis of neuronal synchrony in epilepsy. Adv Neurol 44:583-592.
Wu L, Wells D, Tay J, Mendis D, Abbott M A, Barnitt A, Quinlan E, Heynen A, Fallon J R, Richter J D (1998) CPEB-mediated cytoplasmic polyadenylation and the regulation of experience-dependent translation of α-CaMKII mRNA at synapses. Neuron 21:1129-1139.
Zhang H L, Eom T, Oleynikov Y, Shenoy S M, Liebelt D A, Dictenberg J B, Singer R H, Bassell G J (2001) Neurotrophin-induced transport of a b-actin mRNP complex increases b-actin levels and stimulates growth cone motility. Neuron 31:261-275.

What is claimed is:

1. An isolated antisense molecule comprising a nucleotide sequence targeted to the sequence set forth in SEQ ID NO:1.

2. An isolated antisense molecule comprising a nucleotide sequence targeted to the sequence set forth in SEQ ID NO:2.

3. An isolated antisense molecule comprising the nucleotide sequence set forth in SEQ ID NO:3, said sequence complementary to nucleotides 156-185 of BC200 RNA.

4. An isolated antisense molecule comprising the nucleotide sequence set forth in SEQ ID NO:4, said sequence complementary to nucleotides 158-178 of BC200 RNA.

5. An isolated antisense molecule comprising the nucleotide sequence set forth in SEQ ID NO:5.

6. An isolated nucleic acid molecule comprising the nucleotide sequence set forth in SEQ ID NO:6, complementary to DNA encoding BC200 RNA.

7. The isolated nucleic acid molecule of any one of claims 1-6 admixed with a pharmaceutically acceptable carrier.

8. A method for treating a neurological disorder or cancer in a subject, said method comprising down-regulating BC200 RNA in the subject.

9. The method of claim 8 wherein the down-regulating of BC200 RNA in a subject comprises administering a therapeutically effective amount of a dominant negative mutant of BC200 RNA or a small interfering RNA.

10. The method of claim 8 wherein the down-regulating of BC200 comprises administering a therapeutically effective amount of an antisense molecule targeted to the nucleotide sequence set forth in SEQ ID NO:1 or SEQ ID NO:2.

11. The method of claim 8 wherein the down-regulating of BC200 comprises administering a therapeutically effective amount of at least one of SEQ ID NO:3, SEQ ID NO:4, SEQ ID NO:5, or SEQ ID NO:6.

12. The method of any one claims 8-11 wherein the neurological disorder is at least one of Alzheimer's disease, Fragile X mental retardation syndrome, Down's syndrome and Parkinson's disease.

13. The method of any one of claims 8-11 wherein the cancer is at least one of squamous cell carcinoma of the tongue and lung, epithelial carcinoma of the esophagus, tubular adenocarcinoma of the stomach, breast adenocarcinoma, adenocarcinoma of the lung, mucoepidermoid of the partoid gland, melanoma of the skin, papillary carcinoma of the ovaries, or endothelial adenocarcinoma of the cervix.

14. A method for treating epilepsy in a subject, the method comprising up-regulating BC200 RNA in a patient.

15. The method of claim 14 wherein the up-regulating comprises administering to the patient a therapeutically effective amount of BC200 RNA.

16. The method of claim 14 wherein the up-regulating comprises administering to the patient a gene therapy construct having a DNA or RNA corresponding to BC200 operably linked to a promoter which functions in the cells of the subject.

17. A kit comprising an antisense molecule of any one of claims 1-6 and a pharmaceutically acceptable carrier.

18. The kit of claim 17 wherein the antisense molecule is packaged separately from the pharmaceutically acceptable carrier.

19. The kit of claim 17 wherein the antisense molecule is admixed with the pharmaceutically acceptable carrier.


SEQUENCE LISTING (6 sequences)

SEQ ID NO: 1 - RNA, 200 nt, Homo sapiens
  misc_feature (1)..(2): n at positions 1 and 2 are G and may be present or absent
nnccgggcgcgguggcucacgccuguaaucccagcucucagggaggcuaagaggcgggaggauagcuugagcccaggaguucgagaccugccugggcaauauagcgagaccccguucuccagaaaaaggaaaaaaaaaaacaaaagacaaaaaaaaaauaagcguaacuucccucaaagcaacaaccccccccccccuuu

SEQ ID NO: 2 - RNA, 42 nt, Homo sapiens
uaagcguaacuucccucaaagcaacaaccccccccccccuuu

SEQ ID NO: 3 - DNA, 30 bp, Artificial Sequence
  oligonucleotide
ttgttgctttgagggaagttacgcttattt

SEQ ID NO: 4 - DNA, 21 bp, Artificial Sequence
  oligonucleotide
tttgagggaagttacgcttat

SEQ ID NO: 5 - DNA, 22 bp, Artificial Sequence
  oligonucleotide
cctcttagcctccctgagagct

SEQ ID NO: 6 - DNA, 20 bp, Artificial Sequence
  oligonucleotide
ccagctctcagggaggctaa